(19) United States
(12) Patent Application Publication
(10) Pub. No.: US 2005/0261201 A1
(43) Pub. Date: Nov. 24, 2005

(54) Method of reducing C-reactive protein using growth hormone secretagogues

(75) Inventors: William J. Polvino, Tinton Falls, NJ (US); David B. Carpi, Somerset, NJ (US); Roy G. Smith, Houston, TX (US)

Correspondence Address: Richard B. Smith, Esq.;Palmer & Dodge LLP, 111 Huntington Avenue, Boston, MA, 02199, US

(73) Assignee: Rejuvenon Corporation, Bridgewater, NJ (US)

(21) Appl. No.: 11/094,339

(22) Filed: Mar. 30, 2005

Related U.S. Application Data
(60) Provisional application No. 60/557,466, filed on Mar. 30, 2004.

(51) Int. Cl. A61K 38/05; A61K 31/454

(52) U.S. Cl. 514/18; 514/323

(57) ABSTRACT

The present invention relates to a method of reducing C-reactive protein in a subject in need of treatment thereof, wherein the subject is at risk of having or the subject has already had a vascular event or suffering from an inflammatory disease or disorder. In one embodiment, the vascular event is a cardiovascular event (e.g., myocardial infarction). In another embodiment, the vascular event is a cerebrovascular event (e.g., stroke (such as transient ischemic attacks (TIAs)). In yet another embodiment the vascular event is a peripheral vascular event (e.g., intermittent claudication). The method comprises administering a therapeutically effective amount of at least one growth hormone secretagogue compound or a pharmaceutically acceptable salt, hydrate or solvate thereof. The growth hormone secretagogue can be coadministered with a second growth hormone secretagogue, HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

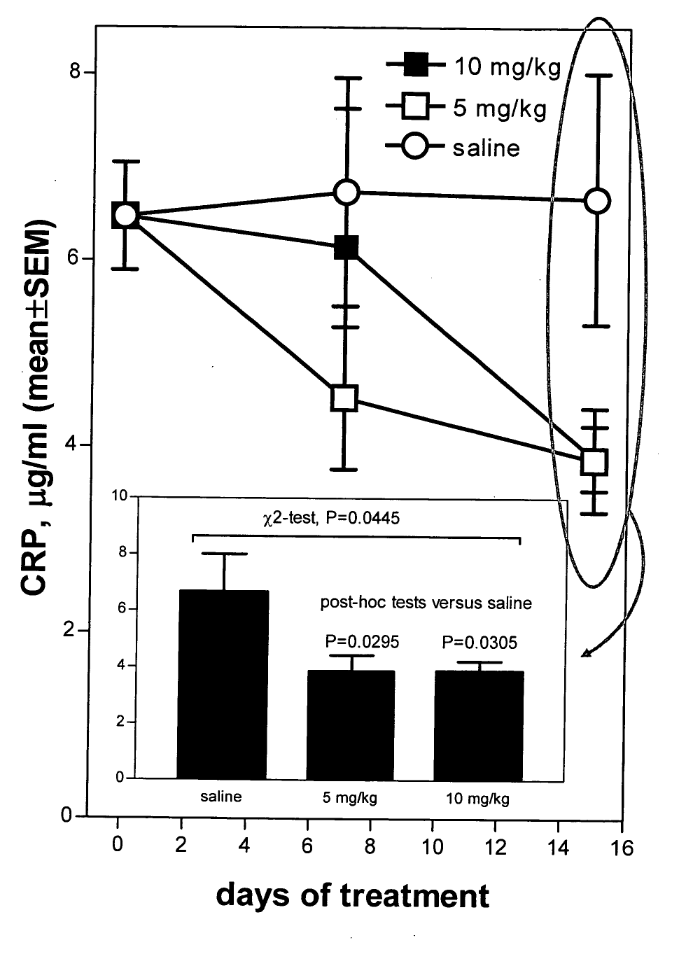

### RELATED APPLICATION

This application claims the benefit of U.S. Provisional Application No. 60/557,466, filed Mar. 30, 2004. The entire teachings of the above application are incorporated herein by reference.

### BACKGROUND OF THE INVENTION

C-reactive protein (also known as CRP and PTX1) is an essential human acute-phase reactant produced in the liver in response to a variety of inflammatory cytokines. The protein, first identified in 1930, is highly conserved and considered to be an early indicator of infectious or inflammatory conditions.

CRP consists of five identical sub-units that contain each 206 amino acids bridged by a single disulfide bond and that aggregate non-covalently into a cyclic pentamer termed pentraxin. The precise biochemical function of CRP as a whole entity is still obscure. Structurally, CRP is a member of the pentraxin family of proteins, which are characterized by a cyclic pentameric structure and radial symmetry. The five identical 24-kDa protomers consist of 206 amino acids, and are noncovalently linked (Lei et al., “Genomic DNA Sequence for Human C-reactive Protein,” J. Biol. Chem., 260(24):13377-83 (1985); Szalai et al., “C-reactive Protein: Structural Biology, Gene Expression, and Host Defense Function,” Immunol. Res., 16(2):127-36 (1997). The genomic DNA sequence for human CRP has been reported by Lei et al., “Genomic DNA Sequence for Human C-reactive Protein,” J. Biol. Chem., 260(24): 13377-83 (1985), as have mutant forms of the protein (Potempa et al., “Stimulation of Megakaryocytopoiesis in Mice by Human Modified C-reactive Protein (mCRP),” Exp. Hematol., 24(2):258-64 (1996) and methods to deliver materials into cells using the mutant protein as a carrier (Potempa et al., “Immunohistochemical Localization of Modified C-reactive Protein Antigen in Normal Vascular Tissue,” Am. J. Med. Sci., 319(2):79-83 (2000). Polypeptides corresponding to amino acids 174-185 of CRP having immunomodulatory activity are disclosed and claimed U.S. Pat. No. 5,783,179 (Nestor et al., 1998). Peptides corresponding to positions 62-71 of human CRP have also been studied for their ability to inhibit the activity of human leukocyte elastase and/or cathepsin G for the treatment of inflammatory conditions and these are disclosed in the PCT Publication WO 99/00418 (Fridkin, 1999).

As an acute phase reactant protein, CRP is usually present in human serum with a concentration of <1 μg/mL. However, C-reactive protein levels can increase up to 100 or even 500 times during acute inflammation. This staggering response is mainly regulated by proinflammatory cytokines, in particular interleukin-6, and is largely unaffected by anti-inflammatory drugs and hormones (Kilpatrick et al., “Molecular Genetics, Structure, and Function of C-reactive Protein,” Immunol Res., 10(1):43-53 (1991). Indeed, in patients with unstable angina with high C-reactive protein levels at discharge, C-reactive protein remains elevated during the follow-up and is associated with high risk of new coronary events, in particular in patients in the upper tertile of C-reactive protein levels (>8.6 μg/mL) (Biasucci et al., “Role of Inflammation in the Pathogenesis of Unstable Coronary Artery Diseases,” Scand. J. Clin. Lab. Invest. Suppl., 230:12-22 (1999). In a recent large prospective study, patients with unstable angina and C-reactive protein levels of >15 μg/mL at discharge had a 3-fold higher risk of coronary events during a 90-day follow-up (Ferreiros et al., “Independent Prognostic Value of Elevated C-reactive Protein in Unstable Angina,” Circulation, 100(19):1958-63 (1999). These results suggest that the proinflammatory effects of C-reactive protein may contribute to the adverse outcome associated with higher levels of this acute phase reactant protein.

Although there is now strong evidence that C-reactive protein is an independent risk factor for ischemic heart disease (Shah, “C-reactive protein: A Novel Marker of Cardiovascular Risk,” Cardiol Rev., 11(4):169-79 (2003); Ridker et al., “C-reactive Protein and Other Markers of Inflammation in the Prediction of Cardiovascular Disease in Women,” N. Engl. J. Med., 342(12):836-43 (2000), the mechanisms underlying this association are not clear. Since inflammatory responses play an important role in the development and evolution of atherosclerosis and may contribute to its thrombotic complications, C-reactive protein may merely be a marker of inflammatory response. Alternatively, C-reactive protein may have a direct role in the pathogenesis of atherosclerosis (Shah, “C-reactive Protein: A Novel Marker of Cardiovascular Risk,” Cardiol Rev., 11(4):169-79 (2003); Lagrand et al., “C-reactive Protein as a Cardiovascular Risk Factor: More Than an Epiphenomenon?,” Circulation., 100(1):96-102 (1999). In view of the above, further research on the role of C-reactive protein in inflammatory response is needed.

### SUMMARY OF THE INVENTION

The present invention relates to a method of reducing C-reactive protein in a subject in need of treatment thereof. The method comprises administering to the subject in need of treatment thereof a therapeutically effective amount of at least one growth hormone secretagogue compound.

In a particular embodiment, the subject in need of treatment thereof is at risk of having a vascular event. In another embodiment, the subject in need of treatment thereof has already had a vascular event.

In one embodiment, the vascular event is a cardiovascular event. In a particular embodiment, the cardiovascular event is a myocardial infarction.

In another embodiment, the vascular event is a cerebrovascular event. In a particular embodiment, the cerebrovascular event is a stroke (such as transient ischemic attacks (TIAs)).

In yet another embodiment, the vascular event is a peripheral vascular event. In a particular embodiment the peripheral vascular event is intermittent claudication.

In a further embodiment, the subject in need of treatment is suffering from an inflammatory disease or disorder.

In a particular embodiment, the growth hormone secretagogue compounds are those described in U.S. Pat. Nos. 6,303,620, 6,576,648, 5,977,178, 6,566,337, 6,083,908, 6,274,584 and Published International Application No. WO 00/01726, the entire content of all of which are incorporated herein by reference.

In a specific embodiment, the growth hormone secretagogue is represented by the structural Formula I: 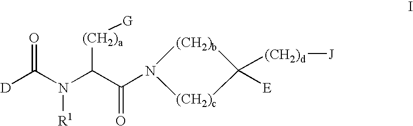
wherein:
R¹ is hydrogen, or C₁₋₆-alkyl optionally substituted with one or more aryl or hetaryl;
a and d are independently 0, 1, 2 or 3;
b and c are independently 0, 1, 2, 3, 4 or 5, provided that b+c is 3, 4 or 5;
D is R²—NH—(CR³R⁴)ₑ—(CH₂)_{f}-M-(CHR⁵)_{g}—(CH₂)ₕ—
   wherein:
R², R³, R⁴ and R⁵ are independently hydrogen or C₁₋₆ alkyl optionally substituted with one or more halogen, amino, hydroxyl, aryl or hetaryl; or
R² and R³ or R² and R⁴ or R³ and R⁴ can optionally form —(CH₂)ᵢ—U—(CH₂)ⱼ—, wherein i and j are independently 1 or 2 and U is —O—, —S— or a valence bond;
h and f are independently 0, 1, 2, or 3;
g and e are independently 0 or 1;
M is a valence bond, —CR⁶═CR⁷—, arylene, hetarylene, —O— or —S—;
R⁶ and R⁷ are independently hydrogen, or C₁₋₆-alkyl optionally substituted with one or more aryl or hetaryl;
G is —O—(CH₂)ₖ—R⁸, 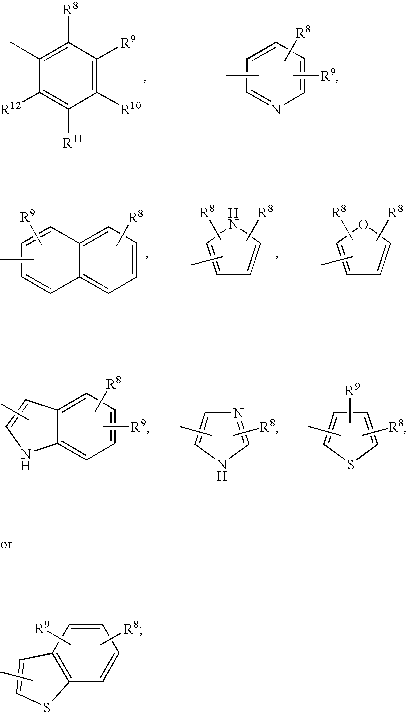
   J is —O—(CH₂)ₗ—R¹³, 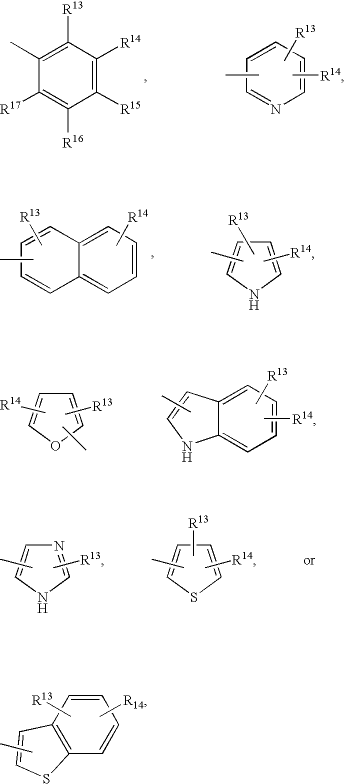
   wherein:
R⁸, R⁹, R¹⁰, R¹¹, R¹², R¹³, R¹⁴, R¹⁵, R¹⁶ and R¹⁷ independently are hydrogen, halogen, aryl, hetaryl, C₁₋₆-alkyl or C₁₋₆-alkoxy;
k and l are independently 0, 1 or 2;
E is —CONR¹⁸ R¹⁹, —COOR¹⁹, —CH₂)ₘ—NR¹⁸SO₂R²⁰, —(CH₂)ₘ—NR¹⁸—COR²⁰, —(CH₂)ₘ—OR¹⁹, —(CH₂)ₘ—OCOR²⁰, —CH(R¹⁸)R¹⁹, —(CH₂)ₘ—NR¹⁸—CS—NR¹⁹R²¹ or —(CH₂)ₘ NR¹⁸—CO—NR¹⁹R²¹; or
E is —CONR²²NR²³R²⁴, wherein R²² is hydrogen, C₁₋₆-alkyl optionally substituted with one or more aryl or hetaryl, or aryl or hetaryl optionally substituted with one or more C₁₋₆-alkyl; R²³ is C₁₋₆-alkyl optionally substituted with one or more aryl or hetaryl, or C₁₋₇-acyl; and R²⁴ is hydrogen, C₁₋₆-alkyl optionally substituted with one or more aryl or hetaryl; or aryl or hetaryl optionally substituted with one or more C₁₋₆-alkyl; or
R²² and R²³ together with the nitrogen atoms to which they are attached can form a heterocyclic system optionally substituted with one or more C₁₋₆-alkyl, halogen, amino, hydroxyl, aryl or hetaryl; or
R²² and R²⁴ together with the nitrogen atoms to which they are attached can form a heterocyclic system optionally substituted with one or more C₁₋₆-alkyl, halogen, amino, hydroxyl, aryl or hetaryl; or
R²³ and R²⁴ together with the nitrogen atom to which they are attached can form a heterocyclic system optionally substituted with one or more C₁₋₆-alkyl, halogen, amino, hydroxyl, aryl or hetaryl;
wherein m is 0, 1, 2 or 3,
R¹⁸, R¹⁹ and R²¹ independently are hydrogen or C₁₋₆-alkyl optionally substituted with halogen, —N(R²⁵)R²⁶, wherein R²⁵ and R²⁶ are independently hydrogen or C₁₋₆ alkyl; hydroxyl, C₁₋₆-alkoxy, C₁₋₆-alkoxycarbonyl, C₁₋₆-alkylcarbonyloxy or aryl;
   or R¹⁹ is 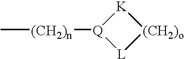
   wherein
Q is —CH< or —N<,
K and L are independently —CH₂—, —CO—, —O—, —S—, —NR²⁷— or a valence bond, where R²⁷ is hydrogen or C₁₋₆ alkyl;
n and o are independently 0, 1, 2, 3 or 4;
R²⁰ is C₁₋₆ alkyl, aryl or hetaryl;
   or a pharmaceutically acceptable salt thereof;
   with the proviso that if M is a valence bond then E is —CONR²²NR²³R²⁴.

The compounds of Formula I are fully described in U.S. Pat. No. 6,303,620 to Hansen, et al., the entire content of which is hereby incorporated by reference.

In another embodiment, the growth hormone secretagogue of Formula I is more specifically represented by the structural Formula II: 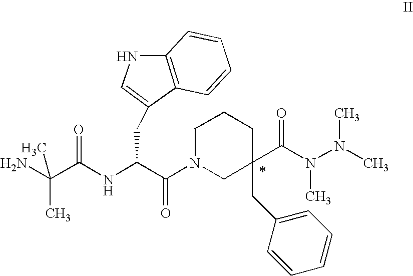
or a pharmaceutically acceptable salt, solvate or hydrate thereof.

The compounds of Formula II are fully described in U.S. Pat. No. 6,303,620 to Hansen, et al., the entire content of which is hereby incorporated by reference.

In yet another embodiment, the growth hormone secretagogue is represented by the structural Formula III: 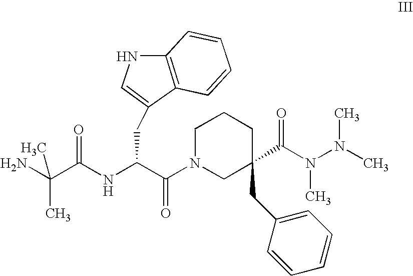
or a pharmaceutically acceptable salt, solvate or hydrate thereof.

The compound of Formula III is fully described in U.S. Pat. No. 6,303,620 to Hansen, et al., the entire content of which is hereby incorporated by reference. The chemical name of the compound of Formula III is 2-Amino-N-{(1R)-2-[3-benzyl-3-(N,N′,N′-trimethylhydrazinocarbonyl)piperidin-1-yl]-1-((1H-indol-3-yl)-2-oxoethyl}-2-methylpropionamide, and is referred to herein as RC-1291.

In a specific embodiment, the growth hormone secretagogue is represented by the structural Formula IV: 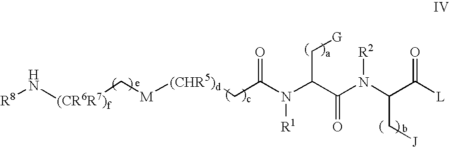
wherein
R¹ is hydrogen or C₁₋₆-alkyl;
R² is hydrogen or C₁₋₆-alkyl;
L is 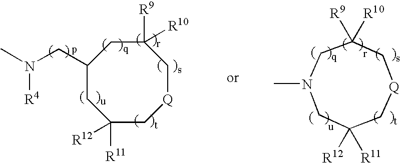
   wherein
R⁴ is hydrogen or C₁₋₆ alkyl;
p is 0 or 1;
q, s, t, u are independently 0, 1, 2, 3, or 4;
r is 0 or 1;
the sumq+r+s+t+u is 0, 1, 2,3, or 4;
R⁹, R¹⁰, R¹¹, and R¹² are independently hydrogen or C₁₋₆alkyl;
Q is >N—R¹³ or 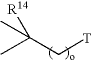
   wherein:
o is 0, 1 or 2;
T is —N(R¹⁵)(R¹⁶) or hydroxyl;
R¹³, R¹⁵, and R¹⁶ are independently hydrogen or C₁₋₆ alkyl;
R¹⁴ is hydrogen, aryl or hetaryl;
G is —O—(CH₂)—R¹⁷, 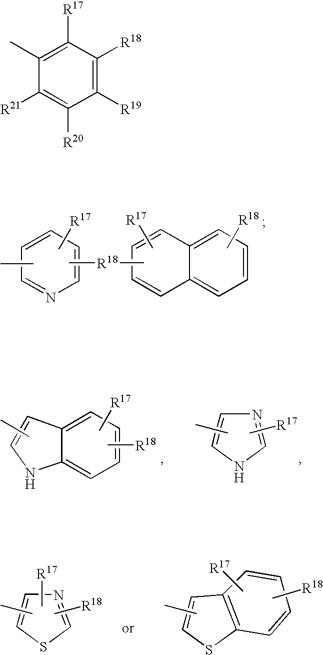
   wherein:
R¹⁷, R¹⁸, R¹⁹, R²⁰ and R²¹ independently are hydrogen, halogen, aryl, hetaryl, C₁₋₆-alkyl or C₁₋₆-alkoxy;
K is 0, 1 or 2;
J is —O—(CH₂)ₗ—R²², 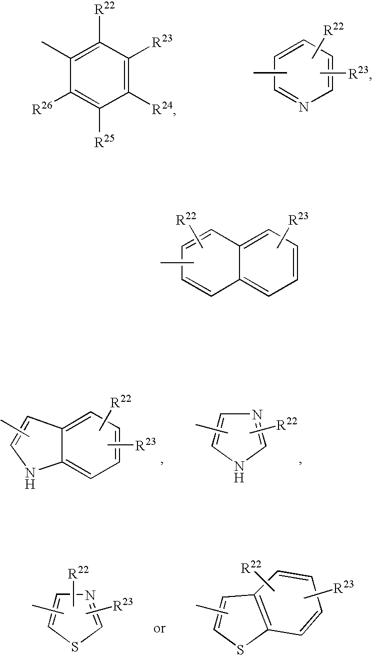
   wherein:
R²², R²³, R²⁴, R²⁵ and R²⁶ independently are hydrogen, halogen, aryl, hetaryl, C₁₋₆-alkyl or C₁₋₆-alkoxy;
1 is 0, 1 or 2;
a is 0, 1, or 2;
b is 0, 1, or 2;
c is 0, 1, or 2;
d is 0 or 1;
e is 0, 1, 2, or 3;
f is 0 or 1;
R⁵ is hydrogen or C₁₋₆-alkyl optionally substituted with one or more hydroxyl, aryl or hetaryl;
R⁶ and R⁷ are independently hydrogen or C₁₋₆-alkyl, optionally substituted with one or more halogen, amino, hydroxyl, aryl, or hetaryl;
R⁸ is hydrogen or C₁₋₆-alkyl, optionally substituted with one or more halogen, amino, hydroxyl, aryl, or hetaryl;
R⁶ and R⁷ or R⁶ and R⁸ or R⁷ and R⁸ can optionally form —(CH₂)ᵢ—U—(CH₂)ⱼ—, wherein i and j independently are 1, 2 or 3 and U is —O—, —S—, or a valence bond;
M is arylene, hetarylene, —O—, —S— or —CR²⁷═CR²⁸—;
R²⁷ and R²⁸ are independently hydrogen or C₁₋₆-alkyl, optionally substituted with one or more aryl or hetaryl;
   or a pharmaceutically acceptable salt thereof.

The compounds of Formula IV are fully described in Published International Application No. WO 00/01726 to Peschke, et al., the entire content of which is hereby incorporated by reference.

In another embodiment, the growth hormone secretagogue of Formula IV is more specifically represented by the structural Formula V: 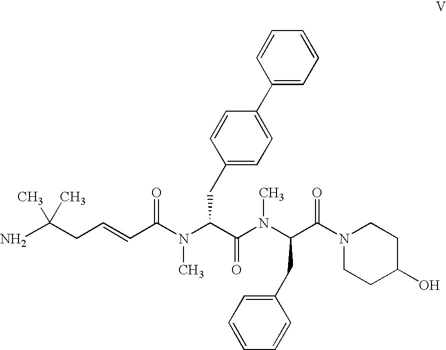
or a pharmaceutically acceptable salt, solvate or hydrate thereof.

The chemical name of the compound of Formula V is (2E)-5-Amino-5-methylhex-2-enoic acid N-((1R)-1-{N-[(1R)-1-benzyl-2-(4-hydroxypiperidin-1-yl)-2-oxoethyl]-N-methylcarbamoyl}-2-(biphenyl-4-yl)ethyl)-N-methylamide, referred to herein as RC-1139.

The compound of Formula V is fully described in Published International Application No. WO 00/01726 to Peschke, et al., the entire content of which is hereby incorporated by reference.

Additional growth hormone secretagogues include compounds which interact with the GHRH receptor. Such compounds include, but are not limited to, GHRH, GHRH (1-29) NH₂, derivatives and analogs thereof having, for example, extended half lives. In addition, sustained release compositions (e.g., patches, microparticles and wafers) containing the GHRH, GHRH (1-29) NH₂, derivatives and analogs thereof are also suitable.

In one embodiment, a growth hormone secretagogue which activates the GHS-R1a receptor can be coadministered with a growth hormone secretagogue which activates the GHRH receptor. In certain embodiments, the coadministration of growth hormone secretagogues acting at the distinct receptors (i.e, GHS-R1a and GHRH) can have enhanced therapeutic effects. In a particular embodiment, the coadministration of secretagogues acting at a distinct receptor further comprises an additional therapeutic agent. Suitable therapeutic agents include, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In another embodiment, the growth hormone secretagogue is coadministered with another therapeutic agent. Suitable therapeutic agents include, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In a particular embodiment, the growth hormone secretagogue compound is coadministered with a HMG CoA reductase inhibitor, such as lovastatin, pravastatin, simvastatin, fluvastatin, atorvastatin, rosuvastatin, cerivastatin or a combination thereof.

In another embodiment, the growth hormone secretagogue is coadministered with an ACAT inhibitor such as avasimibe, FCE 27677, RP 73163 or a combination thereof.

In yet another embodiment, the growth hormone secretagogue is coadministered with a CETP inhibitor such as JTT-705, torcetrapib or a combination thereof.

In a particular embodiment, the growth hormone secretagogue is coadministered with an anti-inflammatory agent such as salicylic acid, aspirin, methyl salicylate, diflunisal, salsalate, olsalazine, sulfasalazine, acetaminophen sulindac, etodolac, tolmetin, ketorolac, diclofenac, ibuprofen, naproxen, fenoprofen, ketoprofen, flurbiprofen, oxaprozin, indomethacin, piroxicam, celecoxib, rofecoxib or a combination thereof.

In another embodiment, the growth hormone secretagogue is coadministered with an ACE inhibitor such as captopril, benazepril, enalapril, fosinopril, lisinopril, quinapril, ramipril, imidapril, perindopril erbumine, trandolapril or a combination thereof.

In yet another embodiment, the growth hormone secretagogue is coadministered with a Beta blocker such as sotalol, timolol, esmolol, careolol, carvedilol, nadolol, propanolol, betaxolol, penbutolol, metoprolol, acebutolol, atenolol, labetolol, pindolol, bisoprolol or a combination thereof.

In a particular embodiment, the growth hormone secretagogue is coadministered with a cholesterol absorption inhibitor such as ezetimibe, tiqueside, pamaqueside or a combination thereof.

In a particular embodiment, the growth hormone secretagogue is coadministered with a nicotonic acid such as niacin, niceritrol or a combination thereof.

In a particular embodiment, the growth hormone secretagogue is coadministered with a fibric acid derivative such as clofibrate, gemfibrozil, fenofibrate, ciprofibrate, bezafibrate or a combination thereof.

In a particular embodiment, the growth hormone secretagogue is coadministered with a bile acid sequestering agent such as cholestyramine, colestipol or a combination thereof.

The invention further relates to pharmaceutical compositions useful for reducing C-reactive protein. The pharmaceutical composition comprises at least one growth hormone secretagogue and optionally a pharmaceutically acceptable carrier. The pharmaceutical composition can comprise a second amount of a second growth hormone secretagogue, a suitable therapeutic agent, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In one embodiment, the pharmaceutical composition can comprise a first amount of a growth hormone secretagogue and a second amount of a second growth hormone secretagogue. The pharmaceutical composition of the present invention can optionally contain a pharmaceutically acceptable carrier. The growth hormone secretagogue and second growth hormone secretagogue can each be present in the pharmaceutical composition in a therapeutically effective amount. In another aspect, said first and second amount can together comprise a therapeutically effective amount. In a particular embodiment, the pharmaceutical composition provides an enhanced therapeutic effect.

In certain embodiments, the pharmaceutical composition comprising a first and second growth hormone secretagogue can contain an additional therapeutic agent selected from the group consisting of, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In one embodiment, the pharmaceutical composition can comprise a first amount of a growth hormone secretagogue and a second amount of a HMG CoA reductase inhibitor. The pharmaceutical composition of the present invention can optionally contain a pharmaceutically acceptable carrier. The growth hormone secretagogue and HMG CoA reductase inhibitor can each be present in the pharmaceutical composition in a therapeutically effective amount. In another aspect, said first and second amount can together comprise a therapeutically effective amount.

In a particular embodiment, the pharmaceutical composition can comprise a first amount of a growth hormone secretagogue and a second amount of an ACAT inhibitor. The pharmaceutical composition of the present invention can optionally contain a pharmaceutically acceptable carrier. The growth hormone secretagogue and ACAT inhibitor can each be present in the pharmaceutical composition in a therapeutically effective amount. In another aspect, said first and second amount can together comprise a therapeutically effective amount.

In another embodiment, the pharmaceutical composition can comprise a first amount of a growth hormone secretagogue and a second amount of a CETP inhibitor. The pharmaceutical composition of the present invention can optionally contain a pharmaceutically acceptable carrier. The growth hormone secretagogue and CETP inhibitor can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of an anti-inflammatory agent. The pharmaceutical composition of the present invention can optionally contain a pharmaceutically acceptable carrier. The growth hormone secretagogue and anti-inflammatory agent can each be present in the pharmaceutical composition in a therapeutically effective amount. In another aspect, said first and second amount can together comprise a therapeutically effective amount.

In a particular embodiment, the pharmaceutical composition can comprise a first amount of a growth hormone secretagogue and a second amount of an ACE inhibitor. The pharmaceutical composition of the present invention can optionally contain a pharmaceutically acceptable carrier. The growth hormone secretagogue and ACE inhibitor can each be present in the pharmaceutical composition in a therapeutically effective amount. In another aspect, said first and second amount can together comprise a therapeutically effective amount.

In another embodiment, the pharmaceutical composition can comprise a first amount of a growth hormone secretagogue and a second amount of a Beta blocker. The pharmaceutical composition of the present invention can optionally contain a pharmaceutically acceptable carrier. The growth hormone secretagogue and Beta blocker can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a cholesterol absorption inhibitor. The pharmaceutical composition of the present invention can optionally contain a pharmaceutically acceptable carrier. The growth hormone secretagogue and cholesterol absorption inhibitor can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a nicotonic acid The pharmaceutical composition of the present invention can optionally contain a pharmaceutically acceptable carrier. The growth hormone secretagogue and nicotonic acid can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a fibric acid derivative. The pharmaceutical composition of the present invention can optionally contain a pharmaceutically acceptable carrier. The growth hormone secretagogue and bile acid sequestering agent can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a bile acid sequestering agent. The pharmaceutical composition of the present invention can optionally contain a pharmaceutically acceptable carrier. The growth hormone secretagogue and bile acid sequestering agent can each be present in the pharmaceutical composition in a therapeutically effective amount. In another aspect, said first and second amount can together comprise a therapeutically effective amount.

The invention further relates to use of a growth hormone secretagogue compound for the manufacture of a medicament for reducing C-reactive protein.

### DETAILED DESCRIPTION OF THE DRAWINGS

The Figure is a plot of CRP serum levels (μg/ml) for CRPtg mice administered saline (n=10), 5 mg/kg of RC-1141 (n=10) or 10 mg/kg of RC-1141 (n=10) versus number of days post-treatment. The results show a significant decrease in CRP serum levels at the 5 mg/kg and 10 mg/kg dose of RC-1141 at day 15 post-administration.

The foregoing and other objects, features and advantages of the invention will be apparent from the following more particular description of preferred embodiments of the invention.

### DETAILED DESCRIPTION OF THE INVENTION

The present invention relates to a method of reducing C-reactive protein in a subject in need of treatment thereof. The method comprises administering to the subject in need of treatment thereof a therapeutically effective amount of at least one growth hormone secretagogue compound. In a particular embodiment, the subject in need of treatment thereof is at risk of having a vascular event. In another embodiment, the subject in need of treatment thereof has already had a vascular event.

In one embodiment, the vascular event is a cardiovascular event. In a particular embodiment the cardiovascular event is myocardial infarction.

In another embodiment, the vascular event is a cerebrovascular event. In a particular embodiment the cerebrovascular event is stroke (such as transient ischemic attacks (TIAs)).

In yet another embodiment, the vascular event is a peripheral vascular event. In a particular embodiment the peripheral vascular event is intermittent claudication.

In a further embodiment, the subject in need of treatment is suffering from an inflammatory disease or disorder.

In one embodiment, a growth hormone secretagogue which activates the GHS-R1a receptor can be coadministered with a growth hormone secretagogue which activates the GHRH receptor. In certain embodiments, the coadministration of growth hormone secretagogues acting at the distinct receptors (i.e, GHS-R1a and GHRH) can have enhanced therapeutic effects. In a particular embodiment, the coadministration of secretagogues acting at a distinct receptor further comprises an additional therapeutic agent. Suitable therapeutic agents include, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In one embodiment, the growth hormone secretagogue is coadministered with another therapeutic agent. Suitable therapeutic agents include, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In a particular embodiment, the growth hormone secretagogue compound is coadministered with a HMG CoA reductase inhibitor, such as lovastatin, pravastatin, simvastatin, fluvastatin, atorvastatin, rosuvastatin, cerivastatin or a combination thereof.

In another embodiment, the growth hormone secretagogue is coadministered with an ACAT inhibitor such as avasimibe, FCE 27677, RP 73163 or a combination thereof.

In yet another embodiment, the growth hormone secretagogue is coadministered with a CETP inhibitor such as JTT-705, torcetrapib or a combination thereof.

In a particular embodiment, the growth hormone secretagogue is coadministered with an anti-inflammatory agent such as salicylic acid, aspirin, methyl salicylate, diflunisal, salsalate, olsalazine, sulfasalazine, acetaminophen sulindac, etodolac, tolmetin, ketorolac, diclofenac, ibuprofen, naproxen, fenoprofen, ketoprofen, flurbiprofen, oxaprozin, indomethacin, piroxicam, celecoxib, rofecoxib or a combination thereof.

In another embodiment, the growth hormone secretagogue is coadministered with an ACE inhibitor such as captopril, benazepril, enalapril, fosinopril, lisinopril, quinapril, ramipril, imidapril, perindopril erbumine, trandolapril or a combination thereof.

In yet another embodiment, the growth hormone secretagogue is coadministered with a Beta blocker such as sotalol, timolol, esmolol, careolol, carvedilol, nadolol, propanolol, betaxolol, penbutolol, metoprolol, acebutolol, atenolol, labetolol, pindolol, bisoprolol or a combination thereof.

In a particular embodiment, the growth hormone secretagogue is coadministered with a cholesterol absorption inhibitor such as ezetimibe, tiqueside, pamaqueside or a combination thereof.

In a particular embodiment, the growth hormone secretagogue is coadministered with a nicotonic acid such as niacin, niceritrol or a combination thereof.

In a particular embodiment, the growth hormone secretagogue is coadministered with a fibric acid derivative such as clofibrate, gemfibrozil, fenofibrate, ciprofibrate, bezafibrate or a combination thereof.

In a particular embodiment, the growth hormone secretagogue is coadministered with a bile acid sequestering agent such as cholestyramine, colestipol or a combination thereof.

Growth Hormone Secretagogues

As used herein, the term growth hormone secretagogues refers to a class of substances (e.g., a molecule, a compound) which activates (promotes at least one function characteristic) of either the growth hormone secretagogue receptor type 1a (GHS-R1a) receptor or the growth hormone releasing hormone (GHRH) receptor. In one embodiment, the growth hormone secretagogue binds to either the GHS-RL a receptor or the GHRH receptor and promotes (induces or enhances) the secretion of growth hormone (i.e., behaves as an agonist).

Suitable growth hormone secretagogues include the both natural ligands of the GHS-R1a receptor (ghrelin) and the GHRH receptor (GHRH(1-44)NH₂) and other substances capable of activating either the GHS-R1a receptor or the GHRH receptor. For example, substances having GHS-R1a receptor agonist activity can be identified using suitable receptor binding assays to assess binding affinity and suitable assays to assess activation of cells expressing the GHS-R1a receptor. For example, the activity of an GHS-R1a agonist can be determined using cultured cells isolated from the anterior pituitary gland expressing the GHS-R1a receptor and assessing the secretion of growth hormone from these cells when exposed to test substance. In addition, activation of the signal transduction pathway in cells expressing GHS-R1a receptor when exposed to test substance can also be assessed to determine agonist activity. Such assays can be found in Published International Application No. WO 00/01726. Similar assays can be used to identify substances having GHRH receptor agonist activity using cultured cells expressing the GHRH receptor.

Growth hormone secretagogue receptor (ghrelin receptor) is referred to as the GHS-R1a (See, Howard et al., Science, 273: 947-977 (1996)). The GHS-R1a receptor is expressed in the anterior hypothalamus, pituitary, pancreas, stomach, suprachiasmatic nucleus, supraoptic nucleus, ventromedical hypothalamus, dentate gyrus, CA2 and CA3 regions of the hippocampal structures, tuberomamillary nucleus, pars compacta of substantia nigra, ventral tegmental area, dorsal raphe nuclei, median raphe nuclei, T-cells and in the thymus. Activation of these receptors in the pituitary induces the secretion of growth hormone. In addition to inducing the secretion of growth hormone, recent studies have shown the growth hormone secretagogues can increase appetite and body weight. At typical doses, growth hormone secretagogues are also known to induce the secretion of IGF-1. In a particular embodiment, the growth hormone secretagogue compounds are those described in U.S. Pat. Nos. 6,303,620, 6,576,648, 5,977,178, 6,566,337, 6,083,908, 6,274,584 and Published International Application No. WO 00/01726, the entire content of all of which are incorporated herein by reference.

Cloning and expression of a GHRH receptor from both the pituitary and anterior pituitary have been reported (Mayo et al., Mol. Endocrinol. 6, 1734-1744 (1992) and Gaylinn et al., Mol. Endocrinol. 7, 77-84 (1993))

In a particular embodiment, the growth hormone secretagogue is represented by the structural Formula I: 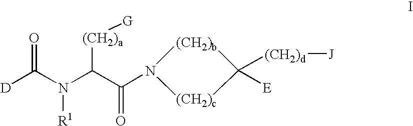
wherein:
R¹ is hydrogen, or C₁₋₆-alkyl optionally substituted with one or more aryl or hetaryl;
a and d are independently 0, 1, 2 or 3;
b and c are independently 0, 1, 2, 3, 4 or 5, provided that b+c is 3, 4 or 5;
D is R² NH—(CR³R⁴)ₑ—(CH₂)_{f}-M-(CHR⁵)_{g}—(CH₂)ₕ—
   wherein:
R², R³, R⁴ and R⁵ are independently hydrogen or C₁₋₆ alkyl optionally substituted with one or more halogen, amino, hydroxyl, aryl or hetaryl; or
R² and R³ or R² and R⁴ or R³ and R⁴ can optionally form —(CH₂)ᵢ—U—(CH₂)ⱼ—, wherein i and j are independently 1 or 2 and U is —O—, —S— or a valence bond;
h and f are independently 0, 1, 2, or 3;
g and e are independently 0 or 1;
M is a valence bond, —CR⁶═CR⁷—, arylene, hetarylene, —O— or —S—;
R⁶ and R⁷ are independently hydrogen, or C₁₋₆-alkyl optionally substituted with one or more aryl or hetaryl;
   G is —O—(CH₂)ₖ—R⁸, 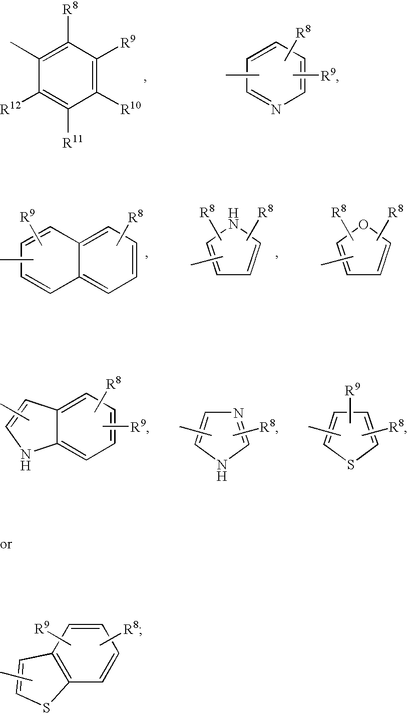
   J is —O—(CH₂)_{f}—R¹³, 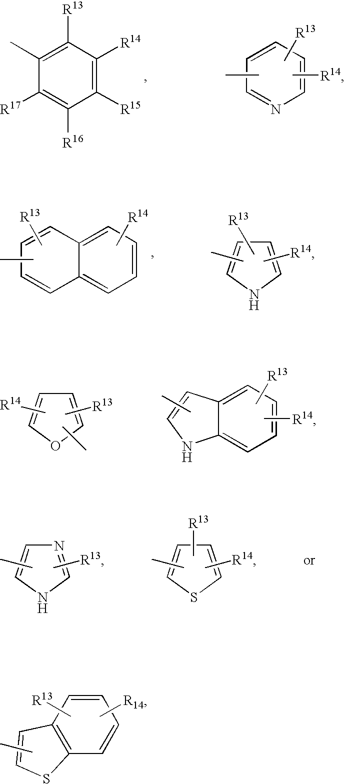
   wherein:
R⁸, R⁹, R¹⁰, R¹¹, R¹², R¹³ R¹⁴ R¹⁵ R¹⁶ and R¹⁷ independently are hydrogen, halogen, aryl, hetaryl, C₁₋₆-alkyl or C₁₋₆-alkoxy;
k and 1 are independently 0, 1 or 2;
E is —CONR¹⁸R¹⁹, —COOR¹⁹, —(CH₂)ₘNR¹⁸ SO₂R²⁰, —(CH₂)ₘ—NR¹⁸—COR²⁰, —(CH₂)ₘ—OR¹⁹, —(CH₂)ₘ—OCOR²⁰, —CH(R¹⁸)R¹⁹, —(CH₂)ₘ—NR¹⁸—CS—NR¹⁹R²¹ or —(CH₂)ₘ—NR¹⁸—CO—NR¹⁹R²¹; or
E is —CONR²²NR²³ R²⁴, wherein R²² is hydrogen, C₁₋₆-alkyl optionally substituted with one or more aryl or hetaryl, or aryl or hetaryl optionally substituted with one or more C₁₋₆-alkyl; R²³ is C₁₋₆-alkyl optionally substituted with one or more aryl or hetaryl, or C₁₋₇-acyl; and R²⁴ is hydrogen, C₁₋₄-alkyl optionally substituted with one or more aryl or hetaryl; or aryl or hetaryl optionally substituted with one or more C₁₋₆-alkyl; or
R²² and R²³ together with the nitrogen atoms to which they are attached can form a heterocyclic system optionally substituted with one or more C₁₋₆-alkyl, halogen, amino, hydroxyl, aryl or hetaryl; or
R²² and R²⁴ together with the nitrogen atoms to which they are attached can form a heterocyclic system optionally substituted with one or more C₁₋₆-alkyl, halogen, amino, hydroxyl, aryl or hetaryl; or
R²³ and R²⁴ together with the nitrogen atom to which they are attached can form a heterocyclic system optionally substituted with one or more C₁₋₆-alkyl, halogen, amino, hydroxyl, aryl or hetaryl;
wherein m is 0, 1, 2 or 3,
R¹⁸, R¹⁹ and R²¹ independently are hydrogen or C₁₋₆-alkyl optionally substituted with halogen, —N(R²⁵)R²⁶, wherein R²⁵ and R²⁶ are independently hydrogen or C₁₋₆ alkyl; hydroxyl, C₁₋₆-alkoxy, C₁₋₆-alkoxycarbonyl, C₁₋₆-alkylcarbonyloxy or aryl;
   or R¹⁹ is 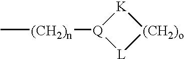
   wherein
Q is —CH< or —N<,
K and L are independently —CH₂—, —CO—, —O—, —S—, —NR²⁷— or a valence bond, where R²⁷ is hydrogen or C₁₋₆ alkyl;
n and o are independently 0, 1, 2, 3 or 4;
R²⁰ is C₁₋₆ alkyl, aryl or hetaryl;
   or a pharmaceutically acceptable salt thereof;
   with the proviso that if M is a valence bond then E is —CONR²²NR²³R²⁴.

In another embodiment, R¹ is C₁₋₆-alkyl. In yet another embodiment, a is 1. In one embodiment, d is 1. In another embodiment, b+c is 4.

In yet another embodiment, D is
R²—NH—(CR³R⁴)ₑ—(CH₂)_{f}-M-(CHR⁵)_{g}—(CH₂)ₕ—
   wherein
R², R³, R⁴ and R⁵ are independently hydrogen or C₁₋₆ alkyl optionally substituted with a halogen, amino, hydroxyl, aryl or hetaryl; or
R² and R³ or R² and R⁴ or R³ and R⁴ can optionally form —(CH₂)ᵢ—U—(CH₂)ⱼ—, wherein i and j are independently 1 or 2 and U is —O—, —S— or a valence bond;
h and f are independently 0, 1, 2, or 3;
g and e are independently 0 or 1;
M is —CR⁶═CR⁷—, arylene, hetarylene, —O— or —S—; and
R⁶ and R⁷ are independently hydrogen, or C₁₋₆-alkyl.

In a further embodiment, D is
R²—NH—(CR³R⁴)ₑ—(CH₂)_{f}-M-(CHR⁵)_{g}—(CH₂)ₕ—
   wherein:
R², R³, R⁴ and R⁵ are independently hydrogen or C₁₋₆ alkyl optionally substituted with a halogen, amino, hydroxyl, aryl or hetaryl; or
   R² and R³ or R² and R⁴ or R³ and R⁴ can optionally form —(CH₂)ᵢ—U—(CH₂)ⱼ—, wherein i and j are independently 1 or 2 and U is —O—, —S— or a valence bond;
   h and f are independently 0, 1, 2, or 3; g and e are independently 0 or 1; M is a valence bond.

In another embodiment, G is 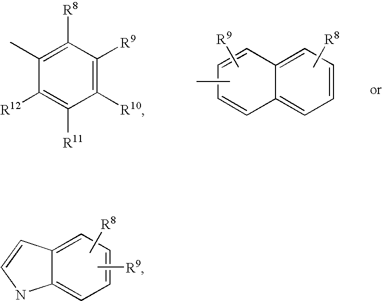
R⁸, R⁹, R¹⁰, R¹¹ and R¹² independently are hydrogen, halogen, aryl, hetaryl, C₁₋₆-alkyl or C₁₋₆ alkoxy; and k is 0, or 2.

In yet another embodiment, J is 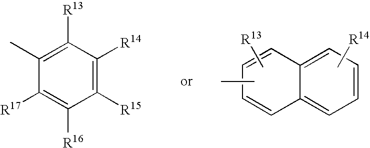
wherein:
R¹³, R¹⁴, R¹⁵, R¹⁶ and R¹⁷ independently are hydrogen, halogen, aryl, hetaryl, C₁₋₆-alkyl or C₁₋₆-alkoxy.

In one embodiment, E is —CONR¹⁸R¹⁹, —COOR¹⁹ or —(CH₂)ₘ—OR¹⁹,

wherein:

m is 0, 1, 2 or 3;
R¹⁸ and R¹⁹ independently are hydrogen or C₁₋₆-alkyl optionally substituted by halogen, —N(R²⁵)R²⁶ wherein R²⁵ and R²⁶ are independently hydrogen or C₁₋₆ alkyl; hydroxyl, C₁₋₆-alkoxy, C₁₋₆-alkoxycarbonyl, C₁₋₆-alkylcarbonyloxy or aryl.

In another embodiment, E is —CONR²²NR²³R²⁴
wherein:
R²² is hydrogen, C₁₋₆-alkyl optionally substituted with an aryl or hetaryl, or aryl or hetaryl optionally substituted with a C₁₋₆-alkyl;
R²³ is C₁₋₆-alkyl optionally substituted with one or more aryl or hetaryl, or C₁₋₇-acyl; and
R²⁴ is hydrogen, C₁₋₆-alkyl optionally substituted with an aryl or hetaryl; or aryl or hetaryl optionally substituted with a C₁₋₆-alkyl; or
R²² and R²³ together with the nitrogen atoms to which they are attached can form a heterocyclic system optionally substituted with a C₁₋₆-alkyl, halogen, amino, hydroxyl, aryl or hetaryl; or R²² and R²⁴ together with the nitrogen atoms to which they are attached can form a heterocyclic system optionally substituted with a C₁₋₆-alkyl, halogen, amino, hydroxyl, aryl or hetaryl; or
R²³ and R²⁴ together with the nitrogen atom to which they are attached can form a heterocyclic system optionally substituted with a C₁₋₆-alkyl, halogen, amino, hydroxyl, aryl or hetaryl.

In a specific embodiment, the growth hormone secretagogue is represented by the structural Formula II: 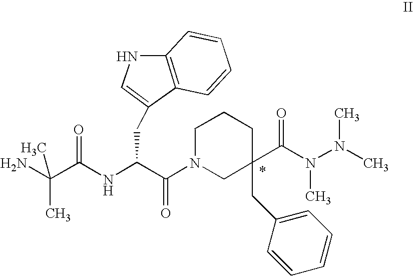
and pharmaceutically acceptable salts thereof.

In a preferred embodiment, the compound of Formula II has the (R) configuration at the chiral carbon designated by the asterix (*) in Formula II. The chemical name of the compound of Formula II having the (R) configuration at the designated chiral carbon is: 2-Amino-N-{(1R)-2-[3-benzyl-3-(N,N′,N′-trimethylhydrazinocarbonyl)piperidin-1-yl]-1-((1H-indol-3-yl)-2-oxoethyl}-2-methylpropionamide. Represented by structural Formula III: 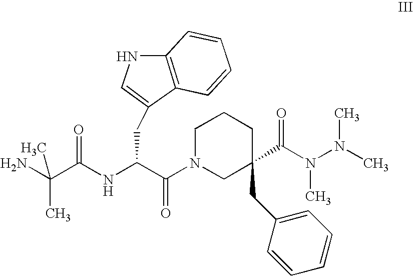
and pharmaceutically acceptable salts thereof.

In a particular embodiment, the growth hormone secretagogue compound is represented by structural Formula IV: 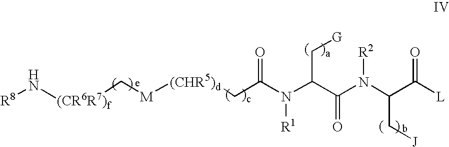
wherein:
R¹ is hydrogen or C₁₋₆-alkyl;
R² is hydrogen or C₁₋₆-alkyl;
L is 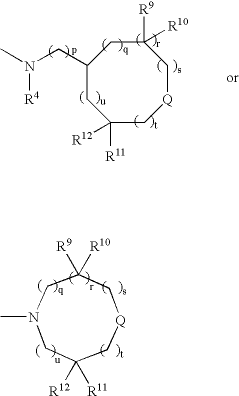
   wherein:
R⁴ is hydrogen or C₁₋₆ alkyl;
p is 0 or 1;
q, s, t, u are independently 0, 1, 2, 3, or 4;
r is 0 or;
the sum q+r+s+t+u is 0, 1, 2,3, or 4;
R⁹, R¹⁰, R¹¹, and R¹² are independently hydrogen or C₁₋₆ alkyl;
   Q is >N—R¹³ or 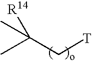
   wherein:
o is 0, 1 or 2;
T is —N(R¹⁵)(R¹⁶) or hydroxyl;
R¹³, R¹⁵, and R¹⁶ are independently hydrogen or C₁₋₆ alkyl;
R¹⁴ is hydrogen, aryl or hetaryl;
G is —O—(CH₂)—R¹⁷, 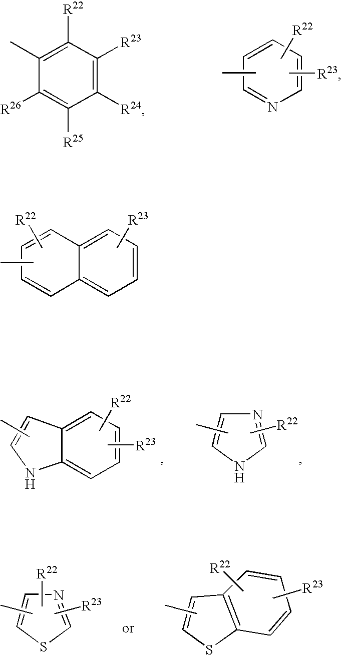
   wherein:
R¹⁷, R¹⁸, R¹⁹, R²⁰ and R²¹ independently are hydrogen, halogen, aryl, hetaryl, C₁₋₆-alkyl or C₁₋₆-alkoxy;
K is 0, 1 or 2;
J is —O—(CH₂)ₗ—R²², 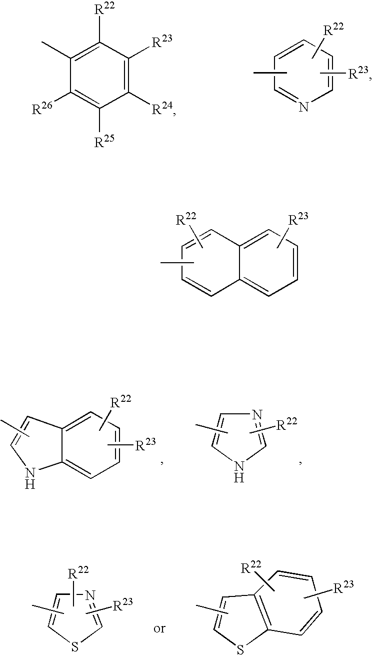
   wherein:
R²², R²³, R²⁴, R²⁵ and R²⁶ independently are hydrogen, halogen, aryl, hetaryl, C₁₋₆-alkyl or C₁₋₆-alkoxy;
1 is 0, 1 or 2;
a is 0, 1, or 2;
b is 0, 1, or 2;
c is 0, 1, or 2;
d is or 1;
e is 0, 1, 2, or 3;
f is 0 or 1;
R⁵ is hydrogen or C₁₋₆-alkyl optionally substituted with one or more hydroxyl, aryl or hetaryl;
R⁶ and R⁷ are independently hydrogen or C₁₋₆-alkyl, optionally substituted with one or more halogen, amino, hydroxyl, aryl, or hetaryl;
R⁸ is hydrogen or C₁₋₆-alkyl, optionally substituted with one or more halogen, amino, hydroxyl, aryl, or hetaryl;
R⁶ and R⁷ or R⁶ and R⁸ or R⁷ and R⁸ can optionally form —(CH₂)ᵢ—U—(CH₂)ⱼ—, wherein i and j independently are 1, 2 or 3 and U is —O—, —S—, or a valence bond;
M is arylene, hetarylene, —O—, —S— or —CR²⁷═CR²⁸—;
R²⁷ and R²⁸ are independently hydrogen or C₁₋₆-alkyl, optionally substituted with one or more aryl or hetaryl;
   or a pharmaceutically acceptable salt thereof.

In another embodiment, R¹ is C₁₋₆-alkyl.

In yet another embodiment, R² is C₁₋₆-alkyl.

In one embodiment, L is 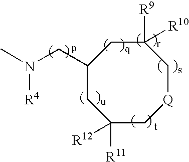
wherein R⁴ is hydrogen or C₁₋₆ alkyl;
p is 0 or 1;
q, s, t, u are independently from each other 0, 1, 2, 3 or 4;
r is 0 or 1;
the sum q+r+s+t+u is 0, 1, 2, 3, or 4;
R⁹, R¹⁰, R¹¹, and R¹² are independently from each other hydrogen or C₁₋₆ alkyl;
Q is >N—R¹³ or 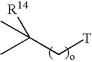
   wherein:
   o is 0, 1 or 2;
   T is —N(R¹⁵)(R¹⁶) or hydroxyl;
   R¹³, R¹⁵, and R¹⁶ are independently from each other hydrogen or C₁₋₆ alkyl; and
   R¹⁴ is hydrogen, aryl or hetaryl.

In another embodiment, L is 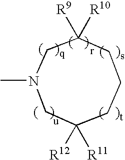
wherein:
q, s, t, u are independently from each other 0, 1, 2, 3 or 4;
   r is 0 or 1;
the sum q+r+s+t+u is 0, 1, 2, 3, or 4;
R⁹, R¹⁰, R¹¹, and R¹² are independently from each other hydrogen or C₁₋₆ alkyl;
Q is >N—R¹³ or 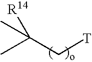
wherein:
o is 0, 1 or 2;
T is —N(R¹⁵)(R¹⁶) or hydroxyl;
R¹³, R¹⁵, and R¹⁶ are independently from each other hydrogen or C₁₋₆ alkyl; and
R¹⁴ is hydrogen, aryl or hetaryl.

In yet another embodiment, G is 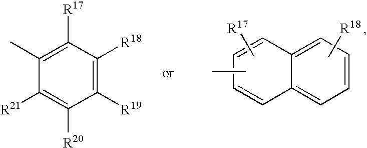
wherein:
R¹⁷, R¹⁸, R¹⁹, R²⁰ and R²¹ independently from each other are hydrogen, halogen, aryl, hetaryl, C₁₋₆-alkyl or C₁₋₆-alkoxy.

In one embodiment, J is 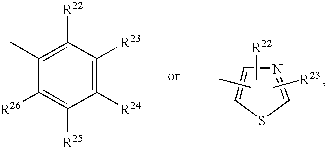
wherein:
R²², R²³, R²⁴, R²⁵ and R²⁶ independently from each other are hydrogen, halogen, aryl, hetaryl, C₁₋₆-alkyl or C₁₋₆-alkoxy.

In another embodiment, M is arylene or —CR²⁷═CR²⁸—, wherein R²⁷ and R²⁸ independently from each other hydrogen or C₁₋₆-alkyl, optionally substituted with aryl or hetaryl.

In yet another embodiment, R⁶ and R⁷ independently from each other are hydrogen or C₁₋₆-alkyl.

In yet another embodiment, R⁶ and R⁷ form —(CH₂)ᵢ—U—(CH₂)ⱼ—, wherein i and j independently from each other are 1, 2 or 3 and U is —O—, —S—, or a valence bond.

In one embodiment, R⁸ is hydrogen or C₁₋₆-alkyl.

In another embodiment, the growth hormone secretagogue compound is represented by the structural Formula V. The chemical name of the compound of Formula V is (2E)-5-Amino-5-methylhex-2-enoic acid N-((1R)-1-{N-[(1R)-1-benzyl-2-(4-hydroxypiperidin-1-yl)-2-oxoethyl]-N-methylcarbamoyl}-2-(biphenyl-4-yl)ethyl)-N-methylamide, also referred to herein as RC-1139. The RC-1139 is represented by structural Formula V: 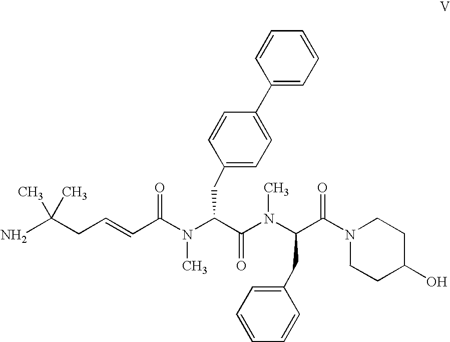
and pharmaceutically acceptable salts thereof.

Other compounds of interest include the following:
1-{(2R)-2-[N-((2E)-5-Amino-5-methylhex-2-enoyl)-N-methylamino]-3-(2-naphthyl)propionyl}-4-benzylpiperidine-4-carboxylic acid methylamide, 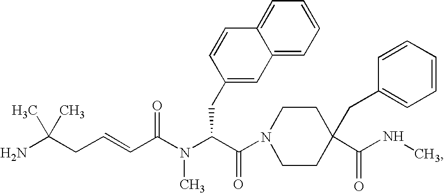
1-{(1R)-2-[N-((2E)-5-Amino-3,5-dimethylhex-2-enoyl)-N-methylamino]-3-(2-naphthyl)propionyl}-4-benzylpiperidine-4-carboxylic acid methylamide 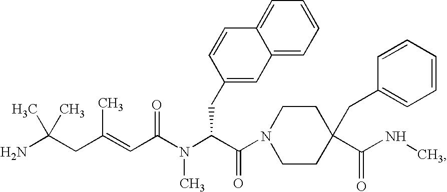
1-{(2R)-2-[N-((2E)-5-Amino-5-methylhex-2-enoyl)-N-methylamino]-3-(biphenyl-4-yl)propionyl}4-benzylpiperidine-4-carboxylic acid methylamide 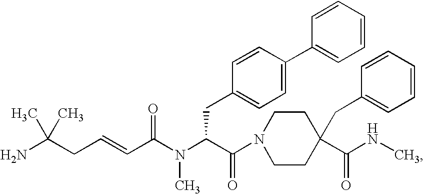
1-{(2R)-2-[N-((2E)-5-Amino-3,5-dimethylhex-2-enoyl)-N-methylamino]-3-(biphenyl-4-yl)propionyl}4-benzylpiperidine-4-carboxylic acid methylamide 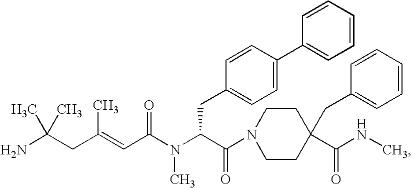
1-((2R)-2-{N-[(2E)4-(1-Aminocyclobutyl)but-2-enoyl]-N-methylamino}-3-(biphenyl-4-yl)propionyl)4-benzylpiperidine-4-carboxylic acid methylamide 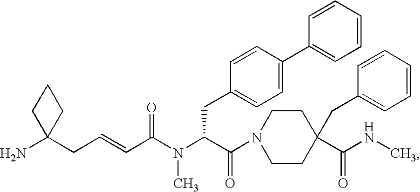
2-Amino-N-[(1R)-2-[4-benzyl-4-(N′,N′-dimethylhydrazinocarbonyl)piperidin-1-yl]-1-((1H-indol-3-yl)methyl)-2-oxoethyl]-2-methylpropionamide 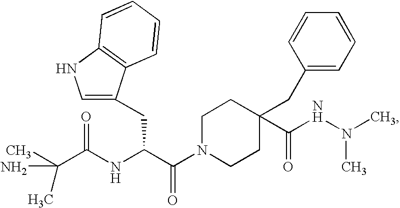
2-Amino-N-{(1R)-2-[(3R)-3-benzyl-3-(N′,N′-dimethyl-hydrazinocarbonyl)-piperidin-1-yl]-1-benzyloxymethyl-2-oxo-ethyl}-2-methyl-propionamide 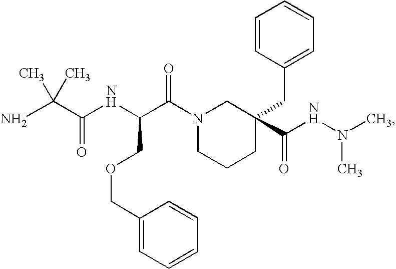
2-Amino-N-[(1R)-2-[(3R)-3-benzyl-3-(N′N′-dimethylhydrazinocarbonyl)-piperidin-1-yl]-1-((1H-indol-3-yl)methyl)-2-oxoethyl]-2-methylpropionamide 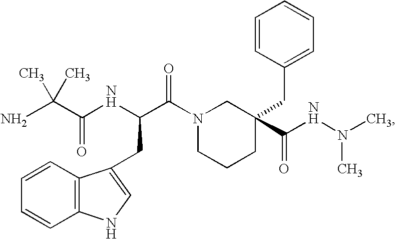
1-{(2R)-2-[N-((2E)-5-Amino-5-methylhex-2-enoyl)-N-methylamino]-3-(biphenyl-4-yl)propionyl}-4-benzylpiperidine-4-carboxylic acid ethyl ester 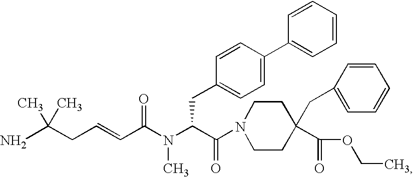
1-{(2R)-2-[N-((2E)-5-Amino-3,5-dimethylhex-2-enoyl)-N-methylamino]-3-(biphenyl-4-yl)propionyl}-4-benzylpiperidine-4-carboxylic acid ethyl ester 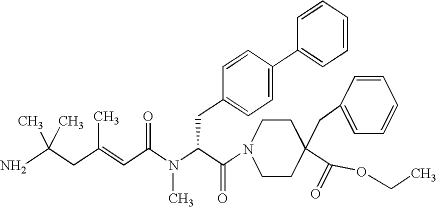
1-{(2R)-2-[N-((2E)-5-Amino-3,5-dimethylhex-2-enoyl)-N-methylamino]-3-(2-naphthyl)propionyl}-4-benzylpiperidine-4-carboxylic acid ethyl ester 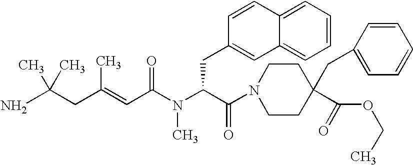
1-{(2R)-2-[N-((2E)-5-Amino-5-methylhex-2-enoyl)-N-methylamino]-3-(2-naphthyl)propionyl}-4-benzylpiperidine-4-carboxylic acid ethyl ester 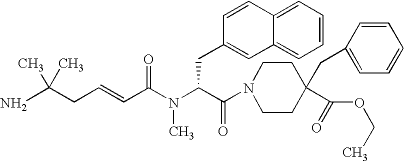
(3S)-1-[(2R)-2-((2E)-5-Amino-5-methylhex-2-enoylamino)-3-(1H-indol-3-yl) propionyl]-3-benzylpiperidine-3-carboxylic acid ethyl ester 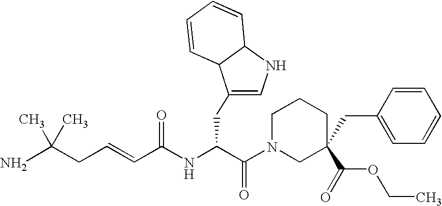
(3S)-1-[(2R)-2-((2E)-5-Amino-3,5-dimethylhex-2-enoylamino)-3-(1H-indol-3-yl) propionyl]-3-benzylpiperidine-3-carboxylic acid ethyl ester 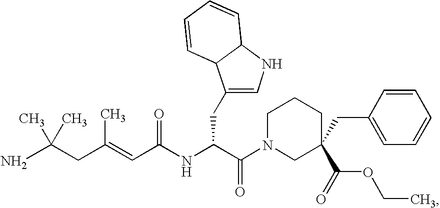
(3S)-1-[(2R)-2-(3-(Aminomethyl)benzoylamino)-3-(1H-indol-3-yl)propionyl]-3-benzylpiperidine-3-carboxylic acid ethyl ester 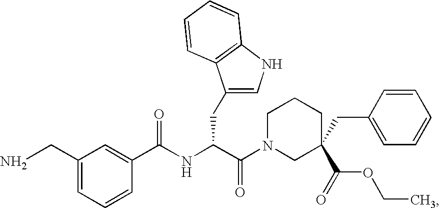
(2E)-5-Amino-5-methylhex-2-enoic acid N-{(1R)-2-[4-benzyl-4-(N′,N′-dimethyl-hydrazinocarbonyl)piperidin-1-yl]-1-((2-naphthyl)methyl)-2-oxoethyl}-N-methylamide 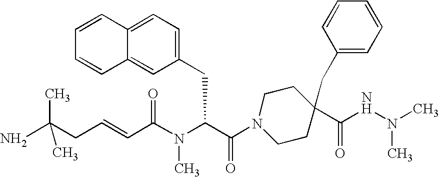
(2E)-5-Amino-5-methylhex-2-enoic acid N-[(1R)-2-[3-benzyl-3-(N′,N′-dimethyl-hydrazinocarbonyl)-piperidin-1-yl]-1-((1H-indol-3-yl)methyl)-2-oxoethyl]amide 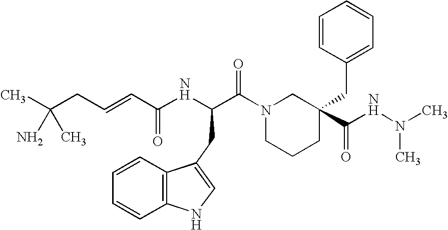
(2E)-5-Amino-5-methylhex-2-enoic acid N-{(1R)-2-[3-benzyl-3-(N′,N′-dimethyl-hydrazinocarbonyl)-piperidin-1-yl]-1-((2-naphthyl)methyl)-2-oxoethyl}-N-methyl-amide 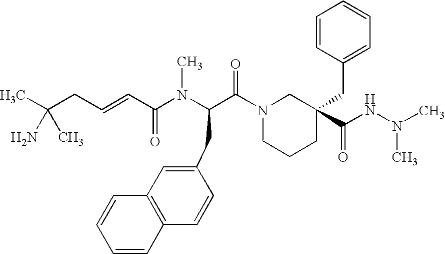
(2E)-5-Amino-5-methylhex-2-enoic acid {(1R)-2-[3-benzyl-3-(N′,N′-dimethyl-hydrazinocarbonyl)piperidin-1-yl]-1-(benzyloxymethyl)-2-oxoethyl}amide 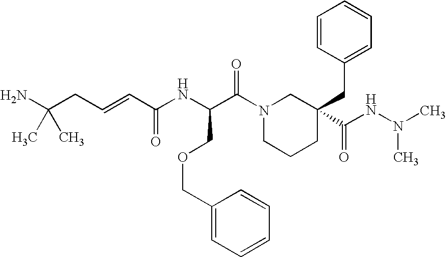
2-Amino-N-{2-[3-benzyl-3-(N′,N′-dimethylhydrazinocarbonyl)piperidin-1-yl]-1-((2-naphthyl)methyl)-2-oxo-ethyl}-2-methyl-propionamide 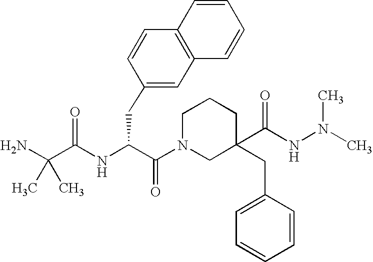
2-Amino-N-{(1R)-2-[3-benzyl-3-(N,N′-dimethylhydrazinocarbonyl)piperidin-1-yl]-1-((biphenyl-4-yl)methyl)-2-oxoethyl}-2-methylpropionamide 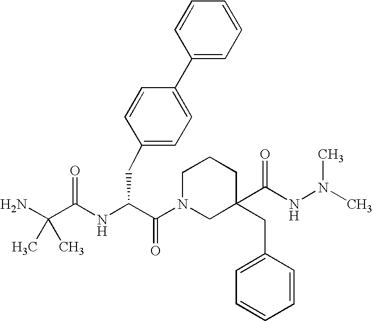
2-Amino-N-{(1R)-2-[3-benzyl-3-(N′,N′-dimethylhydrazinocarbonyl)piperidin-1-yl]-1-((1H-indol-3-yl)methyl)-2-oxoethyl}-2-methylpropionamide 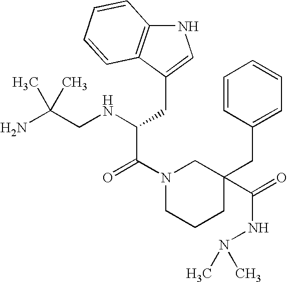
2-Amino-N-{2-[3-benzyl-3-(N′-dimethylhydrazinocarbonyl)piperidin-1-yl]-1-(benzyloxymethyl)-2-oxoethyl}-2-methylpropionamide 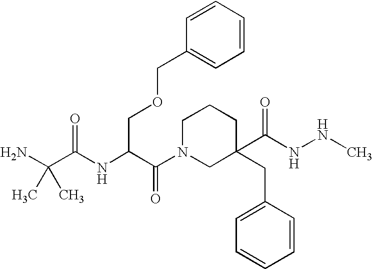
2-Amino-N-{(1R-2-[3-benzyl-3-(N′,N′-dimethylhydrazinocarbonyl)piperidin-1-y l]-1-(benzyloxymethyl)-2-oxoethyl}-2-methylpropionamide 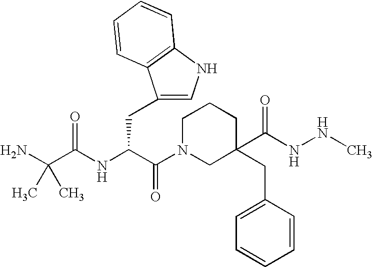
1-[(2R)-2-(2-Amino-2-methylpropionylamino)-3-(1-H-indol-3-yl)propionyl]-3-benzylpiperidine-3-carboxylic acid (pyrrolidin-1-yl)amide 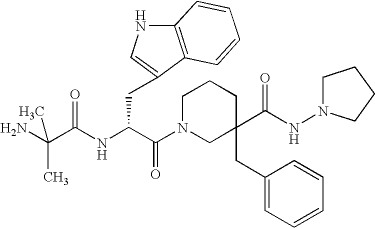
(2E)-5-Amino-5-Methylhex-2-enoic acid N-((1R)-1-{N-[(1R)-1-benzyl-2-(4-((dimethylamino)methyl)piperidin-1-yl)-2-oxoethyl]-N-methylcarbamoyl}-2-(2-naphthyl)ethyl)-N-methylamide 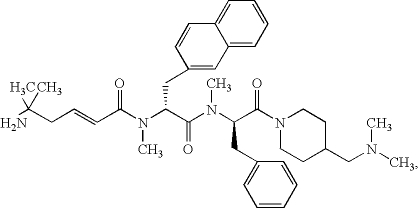
(2E)-5-Amino-5-Methylhex-2-enoic acid N-((1R)-1-{N-[(1R)-1-benzyl-2-((3S)-3-(dimethylaminomethyl)piperidin-1-yl)-2-oxoethyl]-N-methylcarbamoyl}-2-(2-naphthyl)ethyl)-N-methylamide 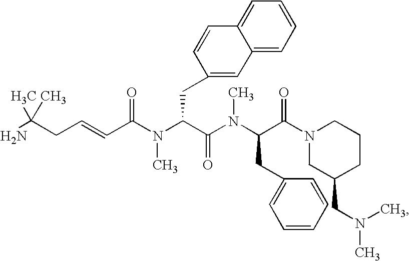
(2E)-4-(1-Aminocyclobutyl)but-2-enoic acid N-((1R)-1-{N-[(1R)-1-benzyl-2-((3S)-3-(dimethylaminomethyl)piperidin-1-yl)-2-oxoethyl]-N-methylcarbamoyl}-2-(2-naphthyl)ethyl)-N-methylamide 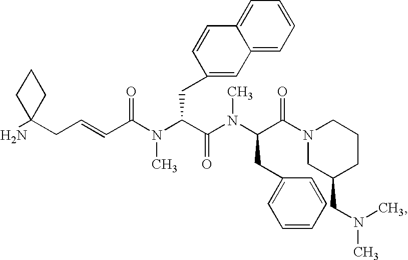
(2E)-5-Amino-5-methylhex-2-enoic acid N-((1R)-1-{N-[(1R)-1-benzyl-2-((2S)-2-((dimethylamino)methyl)pyrrolidin-1-yl)-2-oxoethyl]-N-methylcarbamoyl}-2-(2-naphthyl)ethyl)-N-methylamide 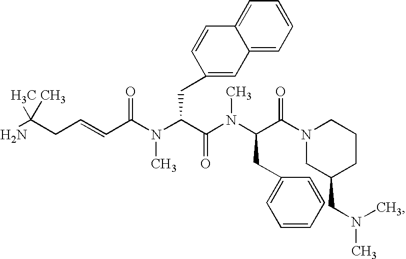
N-((1R)-1-{N-[(1R)-1-Benzyl-2-((2S)-2-((dimethylamino)methyl)pyrrolidin-1-yl)-2-oxoethyl]-N-methylcarbamoyl}-2-(2-naphthyl)ethyl)-N-methyl-3-((methylamino)methyl)benzamide 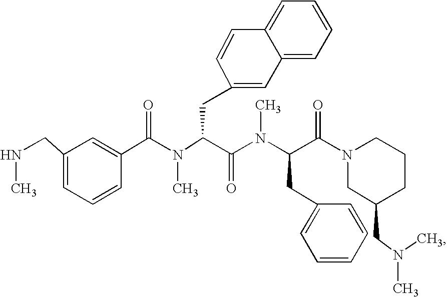
(2E)-5-Amino-5-methylhex-2enoic acid N-((1R-1-{N-[(1R)-1-benzyl-2-(4-(dimethylamino)piperidin-1-yl)-2-oxoethyl]-N-methylcarbamoyl}-2-(2-naphthyl)ethyl)-N-methylamide. 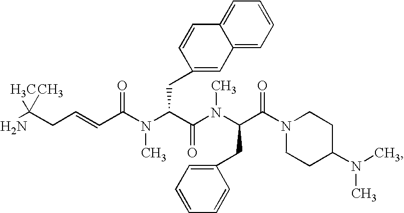
(2E)-5-Amino-5-methylhex-2-enoic acid N-methyl-N-[(1R)-1-(N-methyl-N-{(1R)-1-[N-methyl-N-(1-methylpiperidin-4-yl)carbamoyl]-2-phenylethyl}carbamoyl)-2-(2-naphthyl) ethyl]amide 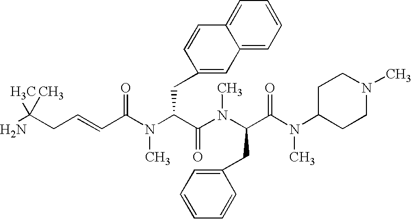
3-Aminomethyl-N-((1R)-1-{N-[(1R)-1-benzyl-2-(4-methylpiperazin-1-yl)-2-oxoethyl]-methylcarbamoyl}-2-(2-naphthyl)ethyl)-N-methylbenzamide 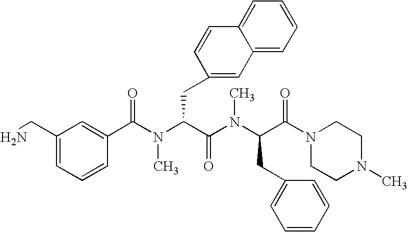
(2E)-5-Amino-5-methylhex-2-enoic acid N-((1R)-1-{N-[(1R)-1-benzyl-2-(4-methylpiperazin-1-yl)-2-oxoethyl]-N-methylcarbamoyl}-2-(2-naphthyl)-N-methylamide 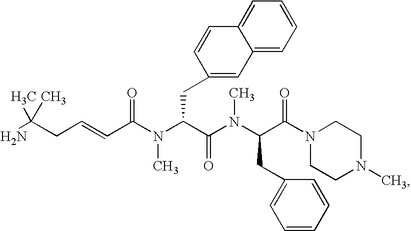
(2E)-5-Amino-5-methylhex-2-enoic acid N-methyl-N-((1R)-1-{N-methyl-N-[(1R)-2-phenyl-1-((2,2,6,6-tetramethylpiperidin-4-yl)carbamoyl)ethyl]carbamoyl}-2-(2-naphthyl)ethyl)amide 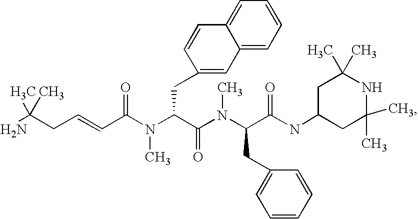
3-Aminomethyl-N-methyl-N-((1R)1-{N-methyl-N-[(1R)-2-phenyl-1-((2,2,6,6-tetramethylpiperidin-4-yl) carbamoyl)ethyl]carbamoyl}-2-(2-naphthyl) ethyl)benzamide 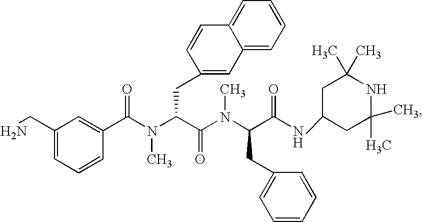
(2E)-5-Amino-3,5-dimethylhex-2-enoic acid N-methyl-N-((1R-1-{N-methyl-N-[(1R)-2-phenyl-1-((2,2,6,6-tetramethylpiperidin-4-yl)carbamoyl)ethyl]carbamoyl}-2-(2-naphthyl)ethyl)amide 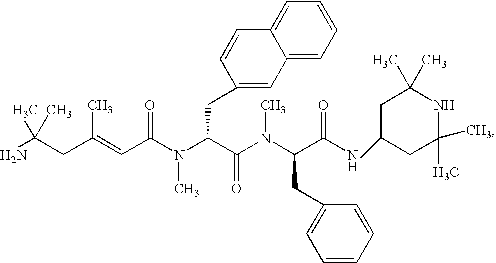
(2E)-4-(1-Aminocyclobutyl)but-2-enoic acid N-((1R)1-{N-[(1R)1-benzyl-2-(4-methylpiperazin-1-yl)-2-oxoethyl]-N-methylcarbamoyl}-2-(2-naphthyl)ethyl)-N-methylamide 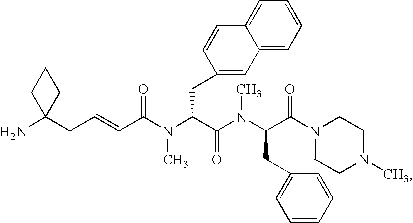
(2E)-5-Amino-3,5-dimethylhex-2-enoic acid N-((1R)1-{N-[(1R)1-benzyl-2-(4-methylpiperazin-1-yl)-2-oxoethyl]-N-methylcarbamoyl}-2-(2-naphthyl)ethyl)-N-methylamide 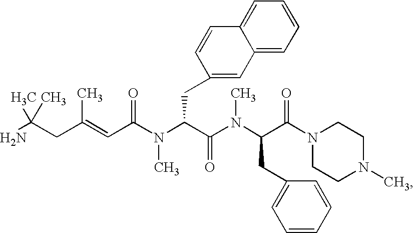
(2E)-4-(1-Aminocyclobutyl)but-2-enoic acid N-((1R)-1-{N-[(1R)-1-benzyl-2-(4-hydroxypiperidin-1-yl)-2-oxoethyl]-N-methylcarbamoyl}-2-(biphenyl-4-yl) ethyl)-N-methylamide 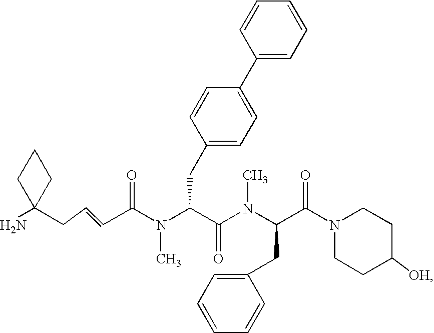
(2E)-5-Amino-3,5-dimethylhex-2enoic acid N-((1R)-1-{N-[(1R)-1-benzyl-2-(4-hydroxypiperidin-1-yl)-2-oxoethyl]-N-methylcarbamoyl}-2-(biphenyl-4-yl)ethyl)-N-methylamide 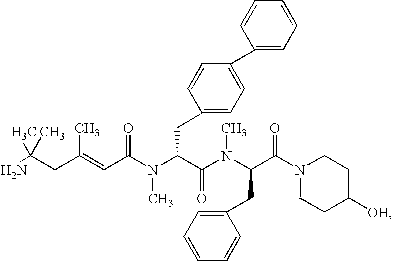
(2E)-5-Amino-5-methylhex-2-enoic acid N-((1R)-1-{N-[(1R)-1-benzyl-2-(4-hydroxypiperidin-1-yl)-2-oxoethyl]-N-methylcarbamoyl}-2-(2-naphthyl)ethyl)-N-methylamide 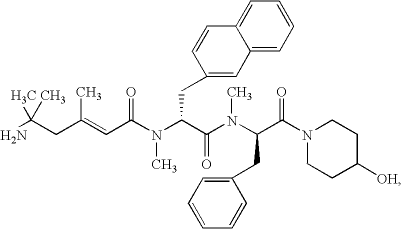
(2E)-5-Amino-3,5-dimethylhex-2-enoic acid N-((1R)-1-{N-[(1R)-1-benzyl-2-(4-hydroxypiperidin-1-yl)-2-oxoethyl]-N-methyl carbamoyl}-2-(2-naphthyl) ethyl)-N-methylamide 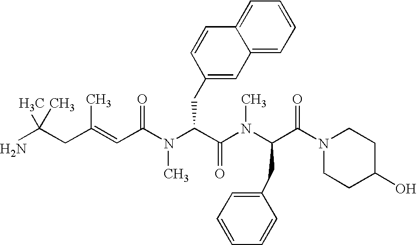
(2E)-4-(1-Aminocyclobutyl)but-2-enoic acid N-((1R)-1-{N-[(1R)-1-benzyl-2-(4-hydroxypiperidin-1-yl)-2-oxoethyl]-N-methylcarbamoyl}-2-(2-naphthyl)ethyl)-N-methylamide 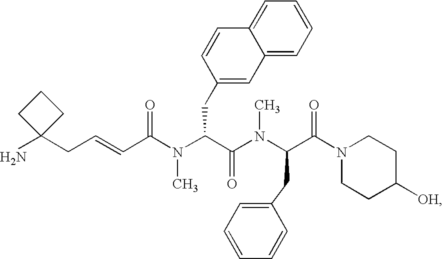
(2E)-5-Amino-5-methylhex-2-enoic acid N-((1R)-1-{N-[(1R)-1-(4-fluorobenzyl)-2-(4-hydroxypiperidin-1-yl)-2-oxoethyl]-N-methylcarbamoyl}-2-(2-naphthyl)ethyl)-N-methylamide 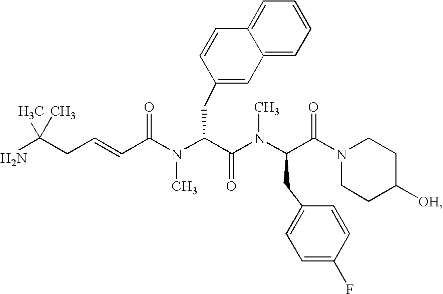
(2E)-5-Amino-3,5-dimethylhex-2-enoic acid N-((1R)-1-{N-[(1R)-1-(4-fluorobenzyl)-2-(4-hydroxypiperidin-1-yl)-2-oxoethyl)-N-methylcarbamoyl}-2-(2-naphthyl)ethyl)-N-methylamide 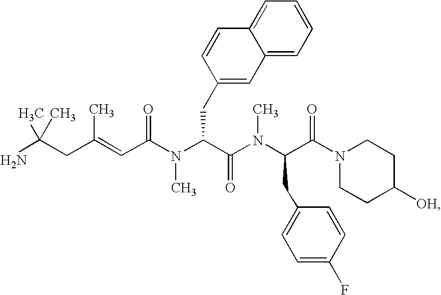
(2E)-5-Amino-5-methylhex-2-enoic acid N-((1R)-1-{N-[(1R)-1-benzyl-2-(4-hydroxy-4-(2-thienyl)piperidin-1-yl)-2-oxoethyl]-N-methylcarbamoyl}-2-(2-naphthyl)ethyl)-N-methylamide 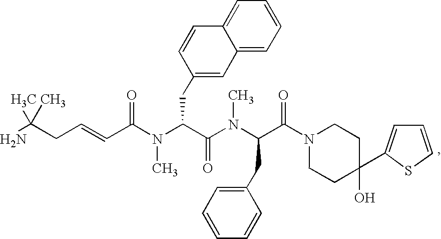
(2E)-5-Amino-5-methylhex-2-enoic acid N-((1R)-1-{N-[(1R)-1-(3-hydroxycyclohexylcarbamoyl)-2-phenylethyl]-N-methylcarbamoyl}-2-(2-naphthyl)ethyl)-N-methylamide 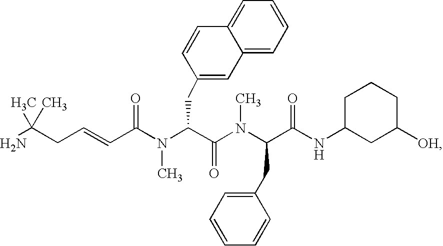
(2E)4-(1-Aminocyclobutyl)but-2-enoic acid N-((1R)-1-{N-[(1R)-1-benzyl-2-(4-(dimethylamino)piperidin-1-yl)-2-oxoethyl]-N-methylcarbamoyl}-2-(2-naphthyl)ethyl)-N-methylamide 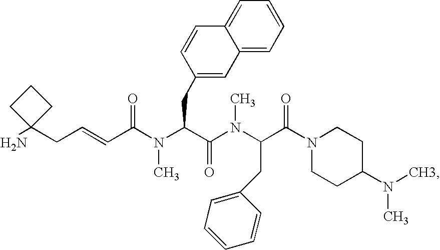
(2E)-5-Amino-5-methylhex-2-enoic acid N-((1R)-1-{N-[(2R)-2-(4-hydroxypiperidin-1-yl)-2-oxo-1-((2-thienyl)methyl)ethyl]-N-methylcarbamoyl}-2-(2-naphthyl)ethyl)-N-methylamide 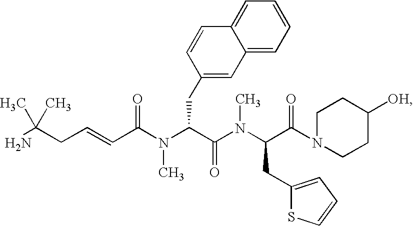
(2E)-5-Amino-3,5-dimethylhex-2-enoic acid N-((1R)-1-{N-[(2R)-2-(4-hydroxypiperidin-1-yl)-2-oxo-1-((2-thienyl)methyl)ethyl]-N-methylcarbamoyl}-2-(2-naphthyl)ethyl)-N-methylamide 2-naphthyl)ethyl)-N-methylamide 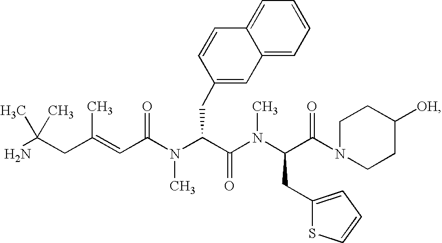
(2E)-5-Amino-5-methylhex-2-enoic acid N-((1R)-2-(biphenyl-4-yl)-1-{N-[(2R)-2-(4-hydroxypiperidin-1-yl)-2-oxo-1-((2-thienyl)methyl)ethyl]-N-methylcarbamoyl}ethyl)-N-methylamide 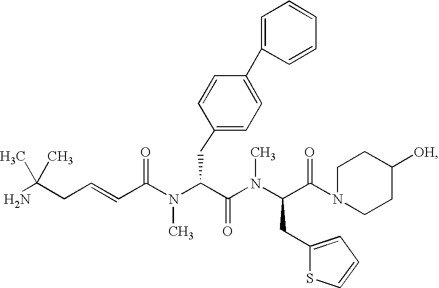
(2E)-5-Amino-3,5-dimethylhex-2-enoic acid N-((1R)-2-(biphenyl-4-yl)-1-{N-[(1R)-2-(4-hydroxypiperidin-1-yl)-2-oxo-1-((2-thienyl)methyl)ethyl]-N-methylcarbamoyl}ethyl)-N-methylamide 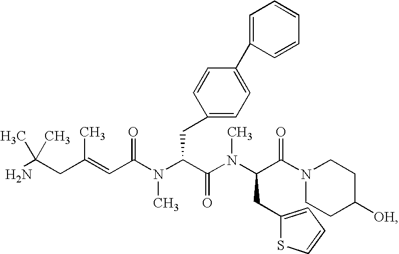
(2E)-5-Methyl-5-(methylamino)hex-2-enoic acid N-((1R)-1-{N-[(1R)-1-benzyl-2-(4-hydroxypiperidin-1-yl)-2-oxoethyl]-N-methylcarbamoyl}-2-(biphenyl-4-yl)ethyl)-N-methylamide 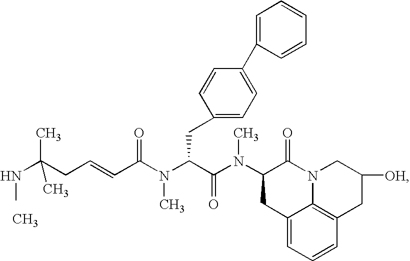
(2E)-4-(1-Aminocyclobutyl)but-2-enoic acid ((1R)-1-{N-[(1R)-1-benzyl-2-(4-hydroxypiperidin-1-yl)-2-oxoethyl]-N-methylcarbamoyl}-2-(biphenyl-4-yl)ethyl)amide 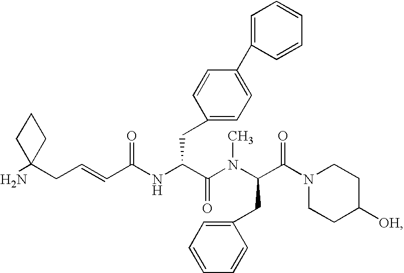
   and pharmaceutically acceptable salts thereof.

In another embodiment, the growth hormone secretagogue is represented by structural Formula VI or a pharmaceutically acceptable salt, solvate or hydrate thereof. The chemical name for the compound represented by structural Formula VI is: (2E)-4-(1-aminocyclobutyl)but-2-enoic acid N-((1R)-1-{N-[(1R)-1-benzyl-2-(4-hydroxypiperidin-1-yl)-2-oxoethyl]-N-methylcarbomoyl}-2-(biphenyl-4-yl)ethyl)-N-methylamide, and is referred to herein as RC-1141.

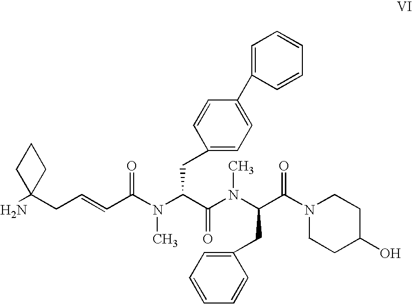

In yet another embodiment, the growth hormone secretagogue is represented by structural Formula VII or a pharmaceutically acceptable salt, solvate or hydrate thereof. The chemical name of the compound represented by structural Formula VII is: (2E)-5-amino-5-methylhex-2-enoic acid N-methyl-N-((1R)-1-{N-methyl-N-[(1R)-2-phenyl-1-(N,N′,N′-trimethylhydrazinocarbonyl)ethyl]carbamoly}-2-(2-naphthyl)ethyl)amide.

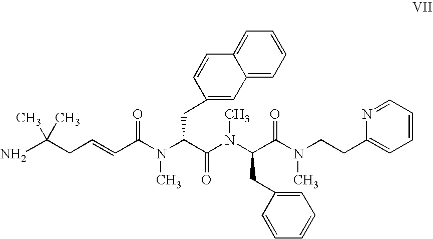

In one embodiment, the growth hormone secretagogue is represented by structural Formula VIII or a pharmaceutically acceptable salt, solvate or hydrate thereof. The chemical name of the compound represented by structural Formula VIII is: (2E)-5-amino-5-methylhex-2-enoic acid N-methyl-N-((1R)-1-{N-methyl-N-[(1R)-2-phenyl-1-(N,N′,N′-trimethylhydrazinocarbony)ethyl]carbamoyl}2-(2-naphthyl)ethyl)amide.

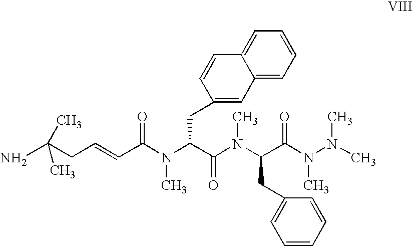

In another embodiment, the growth hormone secretagogue is represented by structural Formula IX or a pharmaceutically acceptable salt, solvate or hydrate thereof. The chemical name for the compound represented by structural Formula IX is: 2-amino-N-(2-(2-(N-((2R)-2-(N-((2E)-5-amino-5-methylhex-2-enoyl)-N-methylamino)-3-(2-napthyl)propionyl)-N-methylamino)ethyl)phenyl)acetamide.

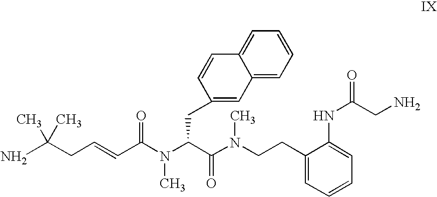

In further embodiments, the growth hormone secretagogue can be selected from GHRH, GHRH (1-29) NH₂, GHRP-1 (Formula X), GHRP-2 (Formula XI), GHRP-6 (Formula XII), NN703 (Formula XIII), Ipamorelin (Formula XIV), Campromorelin (Formula XV), spiroindoline sulfonamides such as MK-677 (Formula XVI), ghrelin, hexarelin (Formula XVII), pyrazolidinon-piperidines such as CP-424,391 (Formula XVIII), oxoindole derivatives such as SM-130686 (Formula XIX, (S)-1-(2-diethylaminoethyl)-4-trifluoromethyl-6-carbamoyl-3-hydroxy-3-(2-chlorophenyl) oxindole), Formula XX and analogs and derivatives of any of the above such as analogs and derivatives with extended half-lives. Sustained release formulations of the above (e.g., pumps, wafers, microparticles and patches) are also suitable. 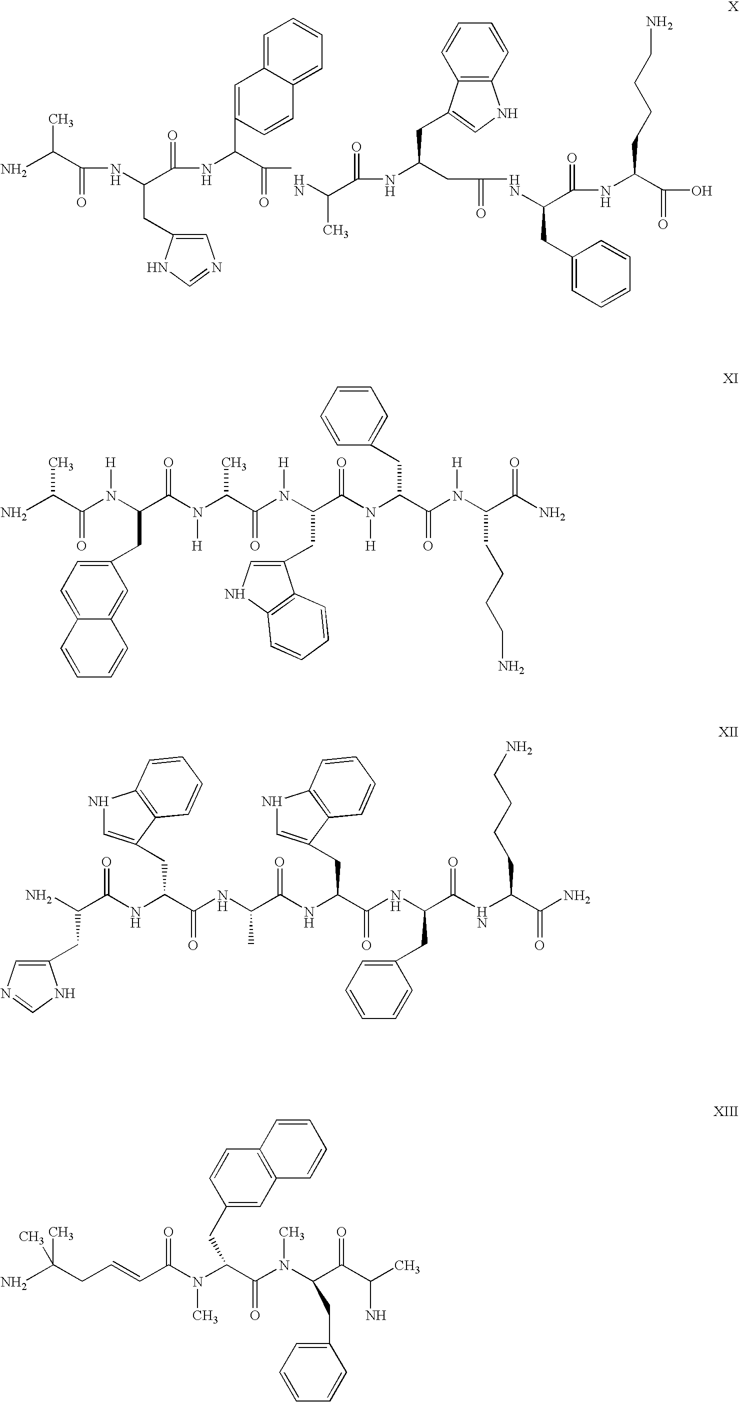 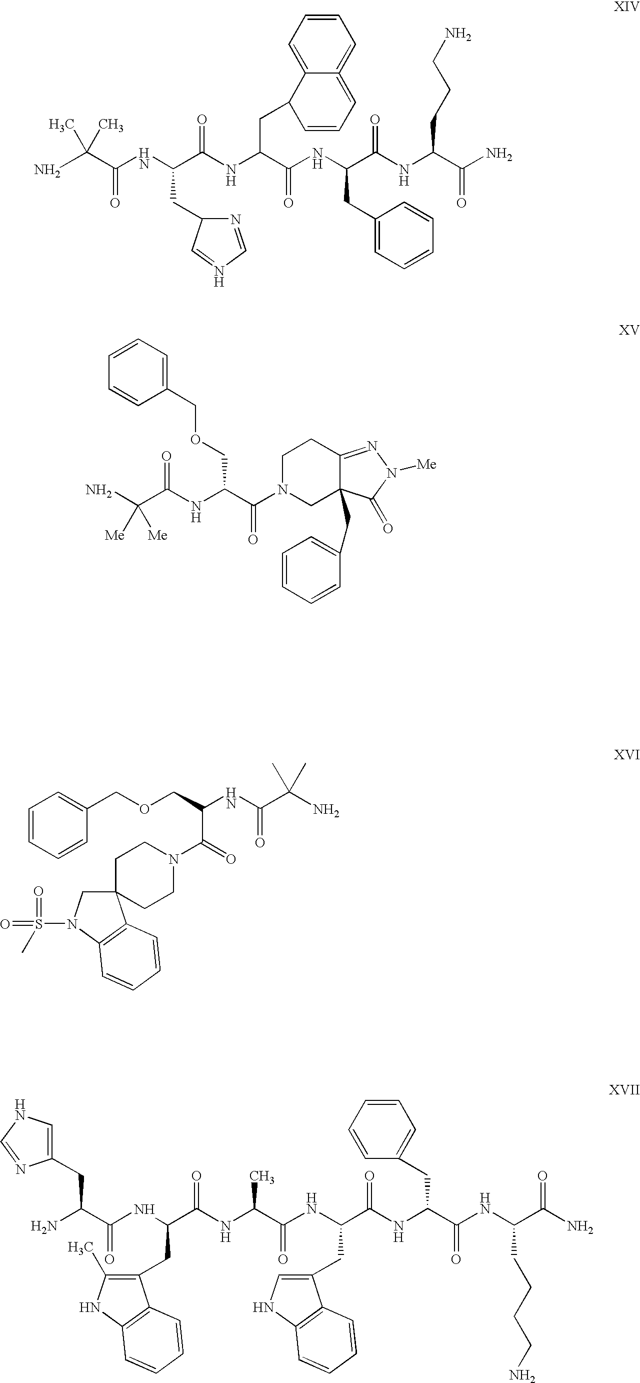 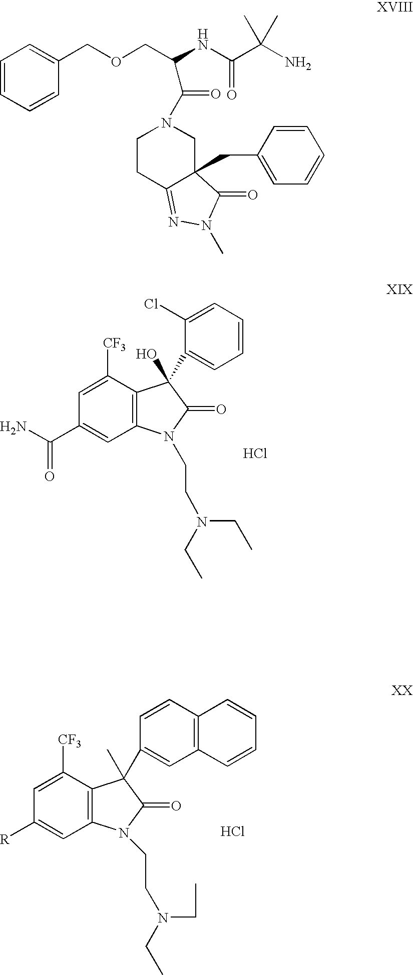
HMG CoA Reductase Inhibitors

A HMG CoA reductase inhibitor is a term of art which refers to a molecule which inhibits the enzyme HMG CoA reductase and, therefore, inhibits the synthesis of cholesterol. This class of compounds inhibits 3-hydroxy-3-methylglutaryl-coenzyme A (HMG CoA) reductase. This enzyme catalyzes the conversion of HMG CoA to mevalonate, which is an early and rate-limiting step in the biosynthesis of cholesterol. As used herein, the HMG CoA reductase inhibitors that are useful in accordance with the methods of the invention satisfy the conventional meaning of this phrase.

The preferred HMG CoA reductase inhibitors that are useful in accordance with the methods and compositions of the invention are statin molecules. These include: lovastatin (MEVACOR®), pravastatin (PRAVACHOL®), simvastatin (ZOCOR®), fluvastatin (LESCOL®), atorvastatin (LIPITOR®), or cerivastatin (BAYCOL®), provided that when the statin molecule is an inhibitor of HMG CoA, it is processed into the corresponding lactone form prior to local administration.

There are a large number of compounds described in the art that have been obtained naturally or synthetically, which have been seen to inhibit HMG CoA reductase, and which form the category of agents useful for practicing the present invention. Traditionally these agents have been used to treat individuals with hypercholesterolemia. Examples include some which are commercially available, such as simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. No. 5,622,985; U.S. Pat. No. 5,135,935; U.S. Pat. No. 5,356,896; U.S. Pat. No. 4,920,109; U.S. Pat. No. 5,286,895; U.S. Pat. No. 5,262,435; U.S. Pat. No. 5,260,332; U.S. Pat. No. 5,317,031; U.S. Pat. No. 5,283,256; U.S. Pat. No. 5,256,689; U.S. Pat. No. 5,182,298; U.S. Pat. No. 5,369,125; U.S. Pat. No. 5,302,604; U.S. Pat. No. 5,166,171; U.S. Pat. No. 5,202,327; U.S. Pat. No. 5,276,021; U.S. Pat. No. 5,196,440; U.S. Pat. No. 5,091,386; U.S. Pat. No. 5,091,378; U.S. Pat. No. 4,904,646; U.S. Pat. No. 5,385,932; U.S. Pat. No. 5,250,435; U.S. Pat. No. 5,132,312; U.S. Pat. No. 5,130,306; U.S. Pat. No. 5,116,870; U.S. Pat. No. 5,112,857; U.S. Pat. No. 5,102,911; U.S. Pat. No. 5,098,931; U.S. Pat. No. 5,081,136; U.S. Pat. No. 5,025,000; U.S. Pat. No. 5,021,453; U.S. Pat. No. 5,017,716; U.S. Pat. No. 5,001,144; U.S. Pat. No. 5,001,128; U.S. Pat. No. 4,997,837; U.S. Pat. No. 4,996,234; U.S. Pat. No. 4,994,494; U.S. Pat. No. 4,992,429; U.S. Pat. No. 4,970,231; U.S. Pat. No. 4,968,693; U.S. Pat. No. 4,963,538; U.S. Pat. No. 4,957,940; U.S. Pat. No. 4,950,675; U.S. Pat. No. 4,946,864; U.S. Pat. No. 4,946,860; U.S. Pat. No. 4,940,800; U.S. Pat. No. 4,940,727; U.S. Pat. No. 4,939,143; U.S. Pat. No. 4,929,620; U.S. Pat. No. 4,923,861; U.S. Pat. No. 4,906,657; U.S. Pat. No. 4,906,624; and U.S. Pat. No. 4,897,402, the disclosures of which patents are incorporated herein by reference.

Additional patents which disclose HMG CoA reductase inhibitors and which are incorporated by reference include: U.S. Pat. No. 6,043,064; Re 36,520; Re 36,481; U.S. Pat. No. 6,001,618; U.S. Pat. No. 5,948,435; U.S. Pat. No. 5,877,208; U.S. Pat. No. 5,792,461; U.S. Pat. No. 5,620,876; U.S. Pat. No. 5,523,460; U.S. Pat. No. 5,475,029; U.S. Pat. No. 5,173,487; U.S. Pat. No. 5,177,080; U.S. Pat. No. 5,189,180; U.S. Pat. No. 5,177,104; U.S. Pat. No. 5,202,327; U.S. Pat. No. 5,250,435; U.S. Pat. No. 5,260,440; U.S. Pat. No. 5,256,692; U.S. Pat. No. 5,266,707; U.S. Pat. No. 5,264,455; U.S. Pat. No. 5,369,123; U.S. Pat. No. 5,371,077; U.S. Pat. No. H1,286; U.S. Pat. No. 5,308,864; U.S. Pat. No. 5,110,825; U.S. Pat. No. 5,106,992; U.S. Pat. No. 5,102,893; U.S. Pat. No. 5,099,035; U.S. Pat. No. 5,098,931; U.S. Pat. No. 5,089,523; U.S. Pat. No. 5,081,136; U.S. Pat. No. 5,075,311; U.S. Pat. No. 5,053,525; U.S. Pat. No. 5,049,696; U.S. Pat. No. 5,032,602; U.S. Pat. No. 5,025,017; U.S. Pat. No. 5,021,453; U.S. Pat. No. 5,010,105; U.S. Pat. No. 5,001,148; U.S. Pat. No. 4,997,837; U.S. Pat. No. 4,997,658; U.S. Pat. No. 4,992,462; U.S. Pat. No. 4,970,231; U.S. Pat. No. 4,970,221; U.S. Pat. No. 4,968,693; U.S. Pat. No. 4,957,971; U.S. Pat. No. 4,957,940; U.S. Pat. No. 4,950,675; U.S. Pat. No. 4,940,800; U.S. Pat. No. 4,937,263; U.S. Pat. No. 4,937,259; U.S. Pat. No. 4,929,620; U.S. Pat. No. 4,923,861; U.S. Pat. No. 4,920,111; U.S. Pat. No. 4,916,162; U.S. Pat. No. 4,906,657; U.S. Pat. No. 4,906,624; U.S. Pat. No. 4,897,402; U.S. Pat. No. 4,885,314; U.S. Pat. No. 4,876,366; U.S. Pat. No. 4,876,279; U.S. Pat. No. 4,868,185; U.S. Pat. No. 4,866,090; U.S. Pat. No. 4,866,068; U.S. Pat. No. 4,864,038; U.S. Pat. No. 4,857,547; U.S. Pat. No. 4,857,546; U.S. Pat. No. 4,855,321; U.S. Pat. No. 4,851,436; U.S. Pat. No. 4,847,306; U.S. Pat. No. 4,808,621; U.S. Pat. No. 4,792,614; U.S. Pat. No. 4,782,084; U.S. Pat. No. 4,772,626; U.S. Pat. No. 4,771,071; U.S. Pat. No. 4,766,145; U.S. Pat. No. 4,761,419; U.S. Pat. No. 4,738,982; U.S. Pat. No. 4,735,958; U.S. Pat. No. 4,719,229; U.S. Pat. No. 4,681,893; U.S. Pat. No. 4,668,699; U.S. Pat. No. 4,665,091; U.S. Pat. No. 4,661,483; U.S. Pat. No. 4,654,363; U.S. Pat. No. 4,647,576; and U.S. Pat. No. 4,567,289.

ACAT Inhibitors

Acyl coenzyme A cholesterol acyltransferase (ACAT) is the enzyme that catalyzes the synthesis of cholesterol ester from cholesterol to play a significant role in the cholesterol metabolism and absorption in gastrointestinal tract. ACAT inhibitors are those such as hereby incorporated by reference as disclosed in, Drugs of the Future 24, 9-15 (1999), (avasimibe; “The ACAT inhibitor, C1-1011 is effective in the prevention and regression of aortic fatty streak area in hamsters”, Nicolosi, et al., Atherosclerosis (Shannon, Irel). (1998), 137(1), 77-85; “The Pharmacological Profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsul-finyl-diphenylimidazole ACAT inhibitor”, Smith, C., et al., Bioorg. Med. Chem. Lett. (1996), 6(1), 47-50; “ACAT inhibitors: physiologic mechanisms for hypolipidemic and anti-atherosclerotic activities in experimental animals”, Krause, et al., Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al., Curr. Med. Chem. (1994), 1(3), 204-25; “Inhibitors of acyl-CoA:cholesterol O-acyl transferase (ACAT) as hypocholesterolemic agents. Development of a series of substituted N-phenyl-N′-[(1-phenylcyclopentyl)methyl]ureas with enhanced hypocholesterolemic activity”, Stout et al., Chemtracts: Org. Chem. (1995), 8(6), 359-62, or TS-962 (Taisho Pharmaceutical Co. Ltd).

CETP Inhibitors

The term CETP inhibitor refers to compounds which inhibit the cholesteryl ester transfer protein (CETP) mediated transport of various cholesteryl esters and triglycerides from high density lipoprotein (HDL) to low density lipoprotein (LDL) and very low density lipoprotein (VLDL). CETP inhibitors include compounds such as torcetrapib (CP-529,414) disclosed in U.S. Pat. No. 6,197,786 and US App. No. 20040053842 (the disclosures of which are incorporated herein by reference) and JTT-705 disclosed in Okamoto et al., Nature 406, 203 (2000), incorporated herein by reference. A variety of these compounds will be known to those skilled in the art U.S. Pat. No. 5,512,548 (the disclosure of which is incorporated herein by reference) discloses certain polypeptide derivatives having activity as CETP inhibitors, while certain CETP-inhibitory rosenonolactone derivatives and phosphate-containing analogs of cholesteryl ester are disclosed in J. Antibiot., 1996; 49(8): 815-816, and Bioorg. Med. Chem. Lett; 1996; 6: 1951-1954, respectively, incorporated herein by reference.

Anti-Inflammatory Agents

The term “NSAID”, as used herein, represents a nonsteroidal anti-inflammatory agent which can be identified as such by the skilled artisan. NSAIDs are known for their inhibition of cyclooxygenases I and II, the enzymes responsible for the biosynthesis of the prostaglandins and certain related autacoids. NSAIDs are known to be antipyretic, analgesic, and antiinflammatory. The term NSAID shall, in addition, refer to any compound acting as a non-steriodal antiinflammatory agent. For example, The Pharmacological Basis of Therapeutics, 9th edition, Macmillan Publishing Co., 1996, pp 617-655, provides well known examples of NSAIDs (the disclosure of which is incorporated herein by reference). The term includes, but is not limited to, salicylic acid derivatives, such as salicylic acid, aspirin, methyl salicylate, diflunisal, salsalate, olsalazine, and sulfasalazine; para-aminophenol derivatives, such as acetaminophen; sulindac; etodolac; tolmetin; ketorolac; diclofenac; propionic acid derivatives, such as ibuprofen, naproxen, fenoprofen, ketoprofen, flurbiprofen, and oxaprozin; acetic acid derivatives, such as indomethacin; enolic acids, such as piroxicam; and cyclooxygenase II inhibitors, such as celecoxib (e.g., CELEBREX®), and rofecoxib (e.g., VIOXX®).

ACE Inhibitors

Angiotensin-converting enzyme inhibitors (ACE) inhibitors are a first class of anti-hypertensives. Such inhibitors include captopril (CAPOTEN®; Bristol-Myers Squibb), benazepril (LOTENSIN®; Novartis), enalapril (VASOTEC®; Merck), fosinopril (MONOPRIL®; Bristol-Myers Squibb), lisinopril (PRINIVIL®; Merck/Zestril.™.; Astra-Zeneca), quinapril (ACCUPRIL®; Parke-Davis) ramipril (ALTACE®; Hoechst Marion Roussel, King Pharmaceuticals), imidapril (not approved for human use in the USA; approved in Japan), perindopril erbumine (ACEON®; Rhone-Polenc Rorer) and trandolapril (MAVIK®; Knoll Pharmaceutical).

Beta Blockers

Beta-blockers are used in the treatment of high blood pressure (hypertension). Such inhibitors include sotalol (BETAPACE®; Berlex Labs), timolol (BLOCADREN®; Merck), esmolol (BREVIBLOC®; Baxter), careolol (CARTROL®; Abbott Labs), carvedilol (COREG®; GlaxoSmithKline), Nadolol (CORGARD®; Bristol-Myers Squibb), propanolol (INDERAL®; Wyeth), propranolol (INDERAL-LA®; Wyeth), betaxolol (KERLONE®), penbutolol (LEVATOL®; Schwarz Pharma), metoprolol (LOPRESSOR®;Novartis), labetolol (NORMODYNE®; Shire), acebutolol (SECTRAL®; Wyeth Ayerst), atenolol (TENORMIN®; Astra Zeneca), metoprolol (TOPROL-XL®; Astra Zeneca), labetolol (TRANDATE®; Prometheus Labs), pindolol (VISKEN®; Novartis), and bisoprolol (ZEBETA®; Merck).

Cholesterol Absorption Inhibitors

Ezetimibe (ZETIA®, Merck Schering-Plough) is the first of a new class of lipid-lowering drugs known as cholesterol absorption inhibitors including tiqueside, pamaqueside all of which are disclosed in U.S. Pat. No. 6,703,386, the disclosure of which is hereby incorporated by reference. Cholesterol absorption inhibitors inhibit the intestinal absorption of dietary and biliary cholesterol, decreasing the delivery of intestinal cholesterol to the liver.

Nicotonic Acid

Nicotinic acid (niacin) is a B-complex vitamin reported as early as 1955 to act as a hypolipidemic agent (R. Altschl, et al., Arch. Biochem. Biophys., 54:558-9 (1955), incorporated herein by reference). It is sometimes used to raise low HDL levels and lower VLDL and LDL levels. As disclosed in U.S. App. No. 20040058908, the disclosure of which is incorporated herein by reference, useful commercial formulations of nicotinic acid include NIACOR®, NIASPAN®, NICOBID®, NICOLAR®, SLO-NIACIN®. Nicotinic acid is contraindicated for patients having hepatic dysfunction, active peptic ulcer, or arterial bleeding. Another compound in this class useful for cardiovascular indications is niceritrol (T. Kazumi et al., Curr. Ther. Res., 55:546-51) (incorporated herein by reference). J. Sasaki et al. (Int. J. Clin. Pharm. Ther., 33 (7):420-26 (1995)), incorporated herein by reference, describes a reduction in cholesterol ester transfer activity by niceritrol monotherapy.

Fibric Acid Derivatives

Fibric acid derivatives comprise another class of drugs which have effects on lipoprotein levels. Among the first of these to be developed was clofibrate, disclosed in U.S. Pat. No. 3,262,850, the disclosure of which is incorporated herein by reference. Clofibrate is the ethyl ester of p-chlorophenoxyisobutyric acid. A widely used drug in this class is gemfibrozil, disclosed in U.S. Pat. No. 3,674,836 (the disclosure of which is incorporated herein by reference). Gemfibrozil frequently is used to decrease triglyceride levels or increase HDL cholesterol concentrations (The Pharmacological Basis of Therapeutics, p. 893). Fenofibrate (U.S. Pat. No. 4,058,552, the disclosure of which is incorporated herein by reference) has an effect similar to that of gemfibrozil, out additionally decreases LDL levels. Ciprofibrate (U.S. Pat. No. 3,948,973, the disclosure of which is incorporated herein by reference) has similar effects to that of fenofibrate. Another drug in this class is bezafibrate (U.S. Pat. No. 3,781,328, the disclosure of which is incorporated herein by reference).

Bile Acid Sequestering Agents

A class of materials which operates by another mechanism to lower LDL cholesterol comprises bile acid sequestering agents. Such agents are typically anion exchange polymers administered orally to a patient. As the agent passes through the gut, anions of bile acids are sequestered by the agent and excreted. Such sequestering has been speculated to prevent reabsorption by the gut, for example the ileum, thereby preventing conversion of the bile acids into cholesterol. One such bile acid sequestering agent is cholestyramine, a styrene-divinylbenzene copolymer containing quaternary ammonium cationic groups capable of binding bile acids. It is believed that cholestyramine binds the bile acids in the intestinal tract, thereby interfering with their normal enterohepatic circulation. This effect is described by Reihnr et al., “Regulation of hepatic cholesterol metabolism in humans: stimulatory effects of cholestyramine on HMG-COA reductase activity and low density lipoprotein receptor expression in gallstone patients”, Journal of Lipid Research, 31:2219-2226 (1990), incorporated herein by reference). Further description of this effect is found in Suckling et al., “Cholesterol Lowering and bile acid excretion in the hamster with cholestyramine treatment”, Atherosclerosis, 89:183-90 (1991), incorporated herein by reference). This results in an increase in liver bile acid synthesis because of the liver using cholesterol as well as an upregulation of the liver LDL receptors which enhances clearance of cholesterol and decreases serum LDL cholesterol levels.

Another bile acid sequestering agent is colestipol, a copolymer of diethylenetriamine and 1-chloro-2,3-epoxypropane. Colestipol is described in U.S. Pat. No. 3,692,895, the disclosure of which is incorporated herein by reference.

Additional bile acid sequestering agents are described in U.S. Pat. No. 5,917,007, U.S. Pat. No. 6,066,678, U.S. Pat. No. 6,433,026, and U.S. Pat. No. 5,703,188, assigned to Genzyme Corp. For example, one such bile acid sequestering agent is 3-methacrylamidopropyltrimethylammon-ium chloride copolymerized with ethylene glycol dimethacrylate to yield a copolymer.

Yet another class materials proposed as bile acid sequestering agents comprises particles comprising amphiphilic copolymers having a crosslinked shell domain and an interior core domain (Patent application no. PCT/US 97/11610, the disclosure of which is incorporated herein by reference). Structures and preparation of such crosslinked amphiphilic copolymers are described in PCT/US97/11345, the disclosure of which is incorporated herein by reference. Such particles have been given the common name of “knedels” (K. B. Thurmond et al., J. Am. Chem. Soc., 118 (30):7239-40 (1996), incorporated herein by reference).

The invention further relates to pharmaceutical compositions useful for reducing C-reactive protein. The pharmaceutical composition comprises at least one growth hormone secretagogue and optionally a pharmaceutically acceptable carrier. The pharmaceutical composition can comprise second amount of a growth hormone secretagogue, a suitable therapeutic agent, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In one embodiment, the pharmaceutical composition can comprise a first amount of a growth hormone secretagogue and a second amount of a second growth hormone secretagogue. The pharmaceutical composition of the present invention can optionally contain a pharmaceutically acceptable carrier. The growth hormone secretagogue and second growth hormone secretagogue can each be present in the pharmaceutical composition in a therapeutically effective amount. In another aspect, said first and second amount can together comprise a therapeutically effective amount. In a particular embodiment, the pharmaceutical composition provides an enhanced therapeutic effect.

In certain embodiments, the pharmaceutical composition comprising a first and second growth hormone secretagogue can contain an additional therapeutic agent selected from the group consisting of, for example, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, a bile acid sequestering agent or a combination thereof.

In one embodiment, the pharmaceutical composition can comprise a first amount of a growth hormone secretagogue and a second amount of a HMG CoA reductase inhibitor. The pharmaceutical composition of the present invention can optionally contain a pharmaceutically acceptable carrier. The growth hormone secretagogue and HMG CoA reductase inhibitor can each be present in the pharmaceutical composition in a therapeutically effective amount. In another aspect, said first and second amount can together comprise a therapeutically effective amount.

In a particular embodiment, the pharmaceutical composition can comprise a first amount of a growth hormone secretagogue and a second amount of an ACAT inhibitor. The pharmaceutical composition of the present invention can optionally contain a pharmaceutically acceptable carrier. The growth hormone secretagogue and ACAT inhibitor can each be present in the pharmaceutical composition in a therapeutically effective amount. In another aspect, said first and second amount can together comprise a therapeutically effective amount.

In another embodiment, the pharmaceutical composition can comprise a first amount of a growth hormone secretagogue and a second amount of a CETP inhibitor. The pharmaceutical composition of the present invention can optionally contain a pharmaceutically acceptable carrier. The growth hormone secretagogue and CETP inhibitor can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of an anti-inflammatory agent. The pharmaceutical composition of the present invention can optionally contain a pharmaceutically acceptable carrier. The growth hormone secretagogue and anti-inflammatory agent can each be present in the pharmaceutical composition in a therapeutically effective amount. In another aspect, said first and second amount can together comprise a therapeutically effective amount.

In a particular embodiment, the pharmaceutical composition can comprise a first amount of a growth hormone secretagogue and a second amount of an ACE inhibitor. The pharmaceutical composition of the present invention can optionally contain a pharmaceutically acceptable carrier. The growth hormone secretagogue and ACE inhibitor can each be present in the pharmaceutical composition in a therapeutically effective amount. In another aspect, said first and second amount can together comprise a therapeutically effective amount.

In another embodiment, the pharmaceutical composition can comprise a first amount of a growth hormone secretagogue and a second amount of a Beta blocker. The pharmaceutical composition of the present invention can optionally contain a pharmaceutically acceptable carrier. The growth hormone secretagogue and Beta blocker can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a cholesterol absorption inhibitor. The pharmaceutical composition of the present invention can optionally contain a pharmaceutically acceptable carrier. The growth hormone secretagogue and cholesterol absorption inhibitor can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a nicotonic acid The pharmaceutical composition of the present invention can optionally contain a pharmaceutically acceptable carrier. The growth hormone secretagogue and nicotinic acid can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a fibric acid derivative. The pharmaceutical composition of the present invention can optionally contain a pharmaceutically acceptable carrier. The growth hormone secretagogue and fibric acid derivative can each be present in the pharmaceutical composition in a therapeutically effective amount. In another aspect, said first and second amount can together comprise a therapeutically effective amount.

In yet another embodiment, the pharmaceutical composition can comprise a first amount of a growth hormone secretagogue and a second amount of a bile acid sequestering agent. The pharmaceutical composition of the present invention can optionally contain a pharmaceutically acceptable carrier. The growth hormone secretagogue and bile acid sequestering agent can each be present in the pharmaceutical composition in a therapeutically effective amount. In another aspect, said first and second amount can together comprise a therapeutically effective amount.

The invention further relates to use of a growth hormone secretagogue compound for the manufacture of a medicament for reducing C-reactive protein.

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species. In a preferred embodiment, the mammal is a human.

Subject at risk of having a vascular event, as used herein, refers to a subject exhibiting characteristics which present a predisposition to a vascular event, for example, a patient with a family history of vascular events or elevated levels of certain markers indicative of increased risk of vascular events, as known in the art, generally requiring a physician's care.

As used herein, treating and treatment refer to reducing (e.g., decreasing) formation of C-reactive protein.

As used herein, therapeutically effective amount refers to an amount sufficient to elicit the desired biological response. In the present invention, the desired biological response is reducing (e.g., decreasing) levels of C-reactive protein. In one embodiment, the desired biological response is reducing (e.g., decreasing) levels of C-reactive protein in a subject at risk of having a vascular event. In another embodiment, the desired biological response is reducing (e.g., decreasing) levels of C-reactive protein the subject who has already had a vascular event. In yet another embodiment, the desired biological response is reducing (e.g., decreasing) levels of C-reactive protein in a subject having an inflammatory disease or disorder.

In one embodiment, the vascular event is a cardiovascular event. In a particular embodiment the cardiovascular event is a myocardial infarction.

In another embodiment, the vascular event is a cerebrovascular event. In a particular embodiment the cerebrovascular event is a stroke (such as transient ischemic attacks (TIAs)).

In yet another embodiment, the vascular event is a peripheral vascular event. In a particular embodiment the peripheral vascular event is intermittent claudication

As used herein, vascular event refers to an event in any of the vasculature.

As used herein, an inflammatory disease/disorder is a disease or disorder caused by inflammation or having an inflammatory component. A non-exclusive list of inflammatory diseases/disorders includes, but is not limited to, the following: inflammatory conditions of a joint, including rheumatoid arthritis (RA) and psoriatic arthritis; systemic lupus erythematosus; Sjorgren's syndrome; lung diseases (e.g., ARDS); acute pancreatitis; ALS; Alzheimer's disease; cachexia/anorexia; asthma; atherosclerosis; chronic fatigue syndrome; diabetes (e.g., insulin diabetes); glomerulonephritis; graft versus host rejection; hemohorragic shock; hyperalgesia; inflammatory bowel disease; multiple sclerosis; myopathies (e.g., muscle protein metabolism); osteoporosis; Parkinson's disease; pain; pre-term labor; psoriasis; septic shock; cardiac, allograft; vasculopathy; side effects from radiation therapy; temporal mandibular joint disease; tumor metastasis; or an inflammatory condition resulting from strain, sprain, cartilage damage, trauma such as bum, orthopedic surgery, infection or other disease processes.

The therapeutically effective amount or dose will depend on the age, sex and weight of the patient, and the current medical condition of the patient. The skilled artisan will be able to determine appropriate dosages depending on these and other factors to achieve the desired biological response. Suitable dosing ranges can be, for example, from about 0.01 mg to about 500 mg per day, for example, from about 0.1 mg per day to 100 mg per day, such as from about 1 mg to about 50 mg per day, for example, from about 5 mg to about 50 mg per day.

Growth hormone levels can be determined using any suitable assay, for example by Growth Hormone, ICMA detailed by ESOTERIX, Inc. (Calabasas Hills, Calif.) using Test Code: 500213. Growth hormone is measured by a two-site immunometric procedure which utilizes monoclonal antibodies to two distinct epitopes of the hGH molecule. One antibody which binds to the N-terminal portion is immobilized on a polystyrene bead and the other which binds to the C-terminus is labeled with acridinium ester. GH from serum samples or standard solutions is immunoextracted by the antibody-coated bead. The bead is then reacted with the acridinium ester-labeled antibody. The antibody binds to the hGH on the bead to form a sandwich. The acridinium ester on this antibody gives off light when treated with hydrogen peroxide and sodium hydroxide. Emitted light is detected by a luminometer. The values obtained with this assay are similar to those obtained with Hybridtech hGH-IRMA, but are approximately 50% of those obtained with conventional RIAs. The use of the two-site methodology ensures that only the intact 22 kilodalton hGH is detected. GH-ICMA exhibits no significant cross-reaction with structural variants of hGH, including the 20 kilodalton splice variant, of hGH₄₄₋₁₉₁ and hGH₁₋₄₃. It has no significant cross-reactivity with other pituitary hormones, such as prolactin, LH, FSH, TSH, or ACTH, nor does it cross-react with hPL.

CRP levels can be determined using any suitable assay, such as, but not limited to, ELISA. Suitable ELISA kits include those detailed by Immuno-Biological Laboratories, Inc. (Hamburg, Germany) (in USA imported and distributed by KMI Diagnostics, Inc., Minneapolis, Minn.) (e.g., Cat. No.: EU 591 31)). Briefly, the assay is a solid phase enzyme-linked immunosorbent (ELISA) based on the sandwich principle. The microtiter wells are coated with an antibody, such as anti-CRP monoclonal antibody HD2-4, directed toward an epitope of the antigen molecule. An aliquot of patient serum is incubated in the coated well with enzyme conjugated second antibody (E-Ab), directed towards a different region of the antigen molecule. After incubation the unbound E-Ab is washed off. The amount of bound E-Ab is proportional to the concentration of antigen in the sample. After adding a substrate solution, such as phosphate-citrate buffer and peroxide, the intensity of color developed is proportional to the antigen concentration in the sample. The measured ODs of the standards are used to construct a calibration curve against which the unknown samples are compared.

Modes of Administration

The compounds for use in the method of the invention can be formulated for administration by any suitable route, such as for oral or parenteral, for example, transdermal, transmucosal (e.g., sublingual, lingual, (trans)buccal), vaginal (e.g., trans- and perivaginally), (intra)nasal and (trans)rectal), subcutaneous, intramuscular, intradermal, intra-arterial, intravenous, inhalation, and topical administration.

Suitable compositions and dosage forms include tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays, dry powders or aerosolized formulations.

It is preferred that the compounds are orally administered. Suitable oral dosage forms include, for example, tablets, capsules or caplets prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated, e.g., to provide for ease of swallowing or to provide a delayed release of active, using suitable methods. Liquid preparation for oral administration can be in the form of solutions, syrups or suspensions. Liquid preparations (e.g., solutions, suspensions and syrups) are also suitable for oral administration and can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Further, the compounds for use in the methods or compositions of the invention can be formulated in a sustained release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation. In addition, implantable pumps are suitable.

As used herein, the term pharmaceutically acceptable salt refers to a salt of a compound to be administered prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

The growth hormone secretagogues disclosed can be prepared in the form of their hydrates, such as hemihydrate, monohydrate, dihydrate, trihydrate, tetrahydrate and the like and as solvates.

It is understood that growth hormone secretagogue compounds can be identified, for example, by screening libraries or collections of molecules using suitable methods. Another source for the compounds of interest are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

Stereochemistry

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When a compound of the present invention has two or more chiral carbons, it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

Coadministration

Administration of a growth hormone secretagogue can take place prior to, after or at the same time as treatment with another therapeutic agent or a different growth hormone secretagogue or both. Additional therapeutic agents include for example, a HMG CoA reductase inhibitor, an ACAT inhibitor, a CETP inhibitor, an anti-inflammatory agent, an ACE inhibitor, a Beta blocker, a cholesterol absorption inhibitor, a nicotinic acid, a fibric acid derivative, a bile acid sequestering agent or a combination thereof. The therapeutic agent can be administered during the period of growth hormone secretagogue administration but does not need to occur over the entire growth hormone secretagogue treatment period.

Variable Definitions

In the above structural formulas and throughout the present specification, the following terms have the indicated meanings:

The C₁₋₆-alkyl, C₁₋₆-alkylene, C₁₋₄-alkyl or C₁₋₄-alkylene groups specified above are intended to include those alkyl or alkylene groups of the designated length in either a linear or branched or cyclic configuration as permitted. Examples of linear alkyl are methyl, ethyl, propyl, butyl, pentyl, and hexyl and their corresponding divalent moieties, such as ethylene. Examples of branched alkyl are isopropyl, sec-butyl, tert-butyl, isopentyl, and isohexyl and their corresponding divalent moieties, such as isopropylene. Examples of cyclic alkyl are C₃₋₆-cycloalkyl such as cyclopropyl, cyclobutyl, cyclopentyl and cyclohexyl and their corresponding divalent moieties, such as cyclopropylene.

The C₁₋₆-alkoxy groups specified above are intended to include those alkoxy groups of the designated length in either a linear or branched or cyclic configuration. Examples of linear alkoxy are methoxy, ethoxy, propoxy, butoxy, pentoxy, and hexoxy. Examples of branched alkoxy are isopropoxy, sec-butoxy, tert-butoxy, isopentoxy, and isohexoxy. Examples of cyclic alkoxy are cyclopropyloxy, cyclobutyloxy, cyclopentyloxy and cyclohexyloxy.

The C₁₋₇-acyl groups specified above are intended to include those acyl groups of the designated length in either a linear or branched or cyclic configuration. Examples of linear acyl are formyl, acetyl, propionyl, butyryl, valeryl, etc. Examples of branched are isobutyryl, isovaleryl, pivaloyl, etc. Examples of cyclic are cyclopentylcarbonyl, cyclohexylcarbonyl, etc.

In the present context, the term “aryl” is intended to include monovalent carbocyclic aromatic ring moieties, being either monocyclic, bicyclic or polycyclic, e.g., phenyl and napthyl, optionally substituted with one or more C₁₋₆-alkyl, C₁₋₆-alkoxy, halogen, amino or aryl.

In the present context, the term “arylene” is intended to include divalent carbocyclic aromatic ring moieties, being either monocyclic, bicyclic or polycyclic, e.g. selected from the group consisting of phenylene and napthylene, optionally substituted with one or more C₁₋₆alkyl, C₁₋₆alkoxy, halogen, amino or aryl.

In the present context, the term “hetaryl” is intended to include monovalent heterocyclic aromatic ring moieties, being either monocyclic, bicyclic or polycyclic, e.g. selected from the group consisting of pyridyl, 1-H-tetrazol-5-yl, thiazolyl, imidazolyl, indolyl, pyrimidinyl, thiadiazolyl, pyrazolyl, oxazolyl, isoxazolyl, oxadiazolyl, thienyl, quinolinyl, pyrazinyl, or isothiazolyl, optionally substituted by one or more C₁₋₆alkyl, C₁₋₆alkoxy, halogen, amino or aryl.

In the present context, the term “hetarylene” is intended to include divalent heterocyclic aromatic ring moieties, being either monocyclic, bicyclic or polycyclic, e.g. selected from the group consisting of pyridinediyl, 1-H-tetrazolediyl, thiazoldiyl, imidazolediyl, indolediyl, pyrimidinediyl, thiadiazolediyl, pyrazolediyl, oxazolediyl, isoxazolediyl, oxadiazolediyl, thiophenediyl, quinolinediyl, pyrazinediyl, or isothiazolediyl, optionally substituted by one or more C₁₋₆alkyl, C₁₋₆alkoxy, halogen, amino or aryl.

In the present context, the term “heterocyclic system” is intended to include aromatic as well as non-aromatic ring moieties, which may be monocyclic, bicyclic or polycyclic, and contain in their ring structure at least one, such as one, two or three, nitrogen atom(s), and optionally one or more, such as one or two, other hetero atoms, e.g. sulpher or oxygen atoms. The heterocyclic system is preferably selected from pyrazole, pyridazine, triazine, indazole, phthalazine, cinnoline, pyrazolidine, pyrazoline, aziridine, dithiazine, pyrrol, imidazol, pyrazole, isoindole, indole, indazole, purine, pyrrolidine, pyrroline, imidazolidine, imidazoline, pyrazolidine, pyrazoline, piperidine, piperazine, indoline, isoindoline, or morpholine.

The term “halogen” is intended to include chlorine (Cl), fluorine (F), bromine (Br) and iodine (I).

Exemplification

The present invention will now be illustrated by the following Example, which is not intended to be limiting in any way.

Study in Mouse Model

The experiment was conducted to determine the effect of the growth hormone secretagogue RC-1141 on CRP serum levels. The compound RC-1141 was tested in C57BL/6J congenic CRP transgenic mice that express human CRP.

Study Design

The establishment of a breeding colony of CRPtg C57BL/6J congenic mice has been described in Szalai, A. et al., “Human C-Reactive Protein is Protective Against Fatal Streptococcus pneumoniae Infection in Transgenic Mice,” J. Immunol. 155:2557-2563 (1995), the entire content of which is hereby incorporated by reference. The breeding colony was established by mating transgenic founder males with normal female C57BL/6J mice. The normal female C57BL/6J mice were obtained from Charles River Laboratories (Boston, Mass.). Transgenic founders were obtained from Dr. Ulrich Ruther (Institut für Molekularbiologie, Medizinische Hochschule, Hannover, Germany), and were descendants of transgenic mice originally described by Ciliberto, G., R. Arcone, E. F. Wagner, U. Rüther, “Inducible and tissue-specific expression of human C-reactive protein in transgenic mice” EMBO J, 6:4017 (1987), the entire content of which is hereby incorporated by reference. These constructs carry a 31-kb ClaI fragment of human genomic DNA consisting of the CRP gene, 17 kb of 5′-flanking sequence, and 11.3 kb of 3′-flanking sequence. The mice were maintained according to protocols established by the Animal Resources Program at the University of Alabama at Birmingham. To minimize genetic variability, transgenic mice were backcrossed with C57BL/6J partners for at least five generations before commencement of the study.

The effect of the growth hormone secretagogue RC-1141 on CRP serum levels in C57BL/6J congenic CRPtg mice was studied. Mice were separated into three (n=3) treatment groups with ten (n=10) mice per/group. The first treatment group was the control group and received vehicle only (100 μl of a 0.9% solution of NaCl). The second treatment group received 5 mg/kg body weight of RC-1141 (“low dose”) in 100 μl of vehicle. The third treatment group received 10 mg/kg body weight of RC-1141 (“high dose”) in 100 μl of vehicle.

Animals were dosed daily by oral gavage for fourteen (n=14) days. Serum was collected on Day 0 (prior to administration of drug or vehicle)), Day 7 and Day 15. All animals were fasted for six hours prior to serum collection.

CRP serum levels were measured using ELISA. The ELISA used sheep anti-human CRP serum (Cappel, Durham, N.C.) and anti-CRP mAb HD2-4 as the capture and detection Ab, respectively, and affinity-purified human CRP as the standard. See Kilpatrick, J. M. et al., “Demonstration of Calcium-induced Conformational Changes(s) in C-reactive Protein by Using Monoclonal Antibodies,” Mol. Immunol., 19:1559 (1982) and Volanakis, J. E. et al., “C-reactive Protein: Purification by Affinity Chromatography and Physiochemical Characterization,” J. Immunol. Methods, 23:285 (1978), the entire contents of which is hereby incorporated by reference. The assay does not detect mouse CRP and has a lower limit of detection of 20 ng of human CRP per ml of mouse serum.

Study Endpoints

CRP serum levels at Day 0, Day 7 and Day 15 are shown in the Figure. Concentration was determined by the ELISA described above. Statistical analysis was done by chi square test.

Results

The data of the Figure show a significant decrease in CRP serum levels at the 5 mg/kg and 10 mg/kg dose of RC-1141 at Day 15 post administration. The decrease in CRP levels seen with RC-1141, is comparable to decreases seen in similar animal models using statins (e.g., atorvastatin) as the administered compound.

While this invention has been particularly shown and described with references to preferred embodiments thereof, it will be understood by those skilled in the art that various changes in form and details may be made therein without departing from the scope of the invention encompassed by the appended claims.

1. A method of reducing C-reactive protein in a subject in need thereof comprising administering a therapeutically effective amount of at least one growth hormone secretagogue compound or a pharmaceutically acceptable salt, hydrate or solvate thereof.

2. The method of claim 1, wherein the subject is at risk of having a vascular event.

3. The method of claim 1, wherein the subject has already had a vascular event.

4. The method of claim 2 or claim 3, wherein the vascular event is a cardiovascular event.

5. The method of claim 4, wherein the cardiovascular event is a myocardial infarction.

6. The method of claim 2 or claim 3, wherein the vascular event is a cerebrovascular event.

7. The method of claim 6, wherein the cerebrovascular event is a stroke.

8. The method of claim 2 or claim 3, wherein the vascular event is a peripheral vascular event.

9. The method of claim 8, wherein the peripheral vascular event is intermittent claudication.

10. The method of claim 1, wherein the growth hormone secretagogue compound is represented by the structural Formula I: 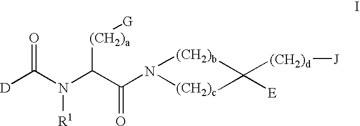
wherein:
R¹ is hydrogen, or C₁₋₆-alkyl optionally substituted with one or more aryl or hetaryl;
a and d are independently 0, 1, 2 or 3;
b and c are independently 0, 1, 2, 3, 4 or 5, provided that b+c is 3, 4 or 5;
D is R²—NH—(CR³R⁴)ₑ—(CH₂)_{f}-M-(CHR⁵)_{g}—(CH₂)ₕ—
wherein:
R², R³, R⁴ and R⁵ are independently hydrogen or C₁₋₆ alkyl optionally substituted with one or more halogen, amino, hydroxyl, aryl or hetaryl; or
R² and R³ or R² and R⁴ or R³ and R⁴ can optionally form —(CH₂)ᵢ—U—(CH₂)ⱼ—, wherein i and j are independently 1 or 2 and U is —O—, —S— or a valence bond;
h and f are independently 0, 1, 2, or 3;
g and e are independently 0 or 1;
M is a valence bond, —CR⁶═CR⁷—, arylene, hetarylene, —O— or —S—;
R⁶ and R⁷ are independently hydrogen, or C₁₋₆-alkyl optionally substituted with one or more aryl or hetaryl;
G is —O—(CH₂)ₖ—R⁸, 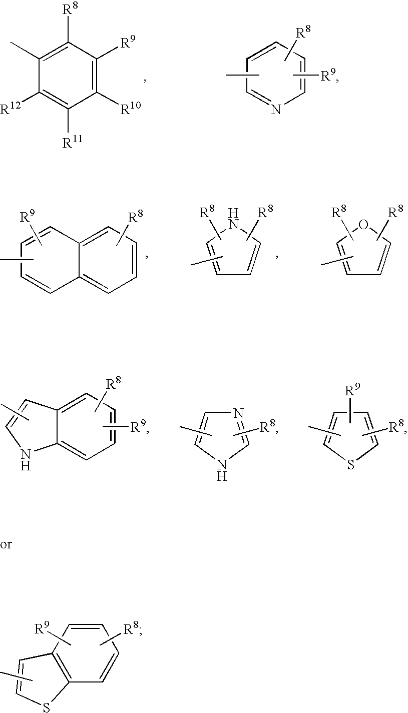
J is —O—(CH₂)ₗ—R¹³, 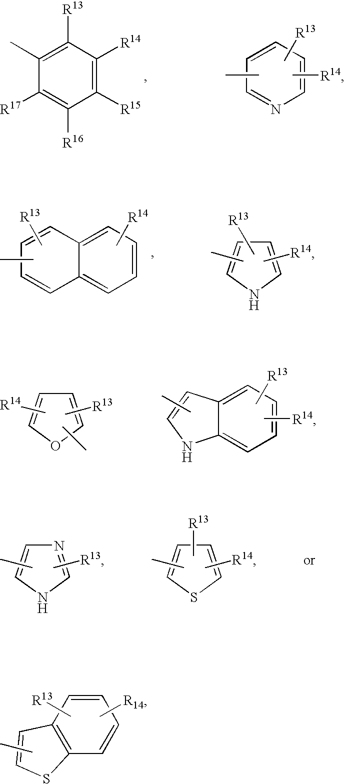
wherein:
R⁸, R⁹, R¹⁰, R¹¹, R¹², R¹³, R¹⁴, R¹⁵, R¹⁶ and R¹⁷ independently are hydrogen, halogen, aryl, hetaryl, C₁₋₆-alkyl or C₁₋₆-alkoxy;
k and 1 are independently 0, 1 or 2;
E is —CONR¹⁸R¹⁹, —COOR¹⁹, —(CH₂)ₘ—NR¹⁸SO₂R²⁰, —(CH₂)ₘ—NR¹⁸—COR²⁰, —(CH₂)ₘ—OR¹⁹, —(CH₂)ₘ—OCOR²⁰, —CH(R¹⁸)R¹⁹, —(CH₂)ₘ—NR¹⁸—CS—NR¹⁹R²¹ or —(CH₂)ₘ—NR¹⁸—CO—NR¹⁹R²¹; or
E is CONR²²NR²³R²⁴, wherein R²² is hydrogen, C₁₋₆-alkyl optionally substituted with one or more aryl or hetaryl, or aryl or hetaryl optionally substituted with one or more C₁₋₆-alkyl; R²³ is C₁₋₆-alkyl optionally substituted with one or more aryl or hetaryl, or C₁₋₇-acyl; and R²⁴ is hydrogen, C₁₋₆-alkyl optionally substituted with one or more aryl or hetaryl; or aryl or hetaryl optionally substituted with one or more C₁₋₆-alkyl; or
R²² and R²³ together with the nitrogen atoms to which they are attached can form a heterocyclic system optionally substituted with one or more C₁₋₆-alkyl, halogen, amino, hydroxyl, aryl or hetaryl; or
R²² and R²⁴ together with the nitrogen atoms to which they are attached can form a heterocyclic system optionally substituted with one or more C₁₋₆-alkyl, halogen, amino, hydroxyl, aryl or hetaryl; or
R²³ and R²⁴ together with the nitrogen atom to which they are attached can form a heterocyclic system optionally substituted with one or more C₁₋₆-alkyl, halogen, amino, hydroxyl, aryl or hetaryl;
wherein m is 0, 1, 2 or 3,
R¹⁸, R¹⁹ and R²¹ independently are hydrogen or C₁₋₆-alkyl optionally substituted with halogen, —N(R²⁵)R²⁶, wherein R²⁵ and R²⁶ are independently hydrogen or C₁₋₆ alkyl; hydroxyl, C₁₋₆-alkoxy, C₁₋₆-alkoxycarbonyl, C₁₋₆-alkylcarbonyloxy or aryl;
or R¹⁹ is 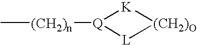
wherein
Q is —CH< or —N<,
K and L are independently —CH₂—, —CO—, —O—, —S—, —NR²⁷— or a valence bond, where R²⁷ is hydrogen or C₁₋₆ alkyl;
n and o are independently 0, 1, 2, 3 or 4;
R²⁰ is C₁₋₆ alkyl, aryl or hetaryl;
or a pharmaceutically acceptable salt thereof;
with the proviso that if M is a valence bond then E is —CONR²²NR²³ R²⁴.

11. The method of claim 10, wherein the growth hormone secretaogue is represented by Formula II: 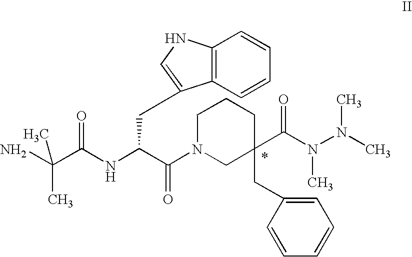
or a pharmaceutically acceptable salt, hydrate or solvate thereof.

12. The method of claim 10, wherein the growth hormone secretagogue is represented by Formula III: 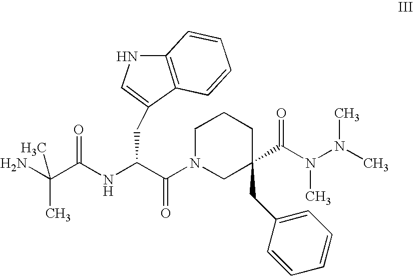
or a pharmaceutically acceptable salt thereof.

13. The method of claim 1, wherein the growth hormone secretagogue is represented by Formula IV: 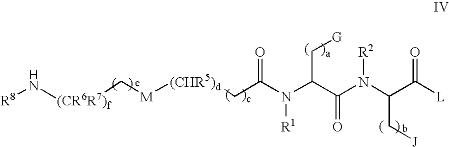
wherein
R¹ is hydrogen or C₁₋₆-alkyl;
R² is hydrogen or C₁₋₆-alkyl;
L is 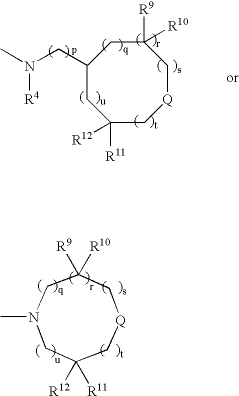
wherein
R⁴ is hydrogen or C₁₋₆ alkyl;
p is 0 or 1;
q, s, t, u are independently 0, 1, 2, 3, or 4;
r is 0 or 1;
the sum q+r+s+t+u is 0, 1, 2,3, or 4;
R⁹, R¹⁰, R¹¹, and R¹² are independently hydrogen or C₁₋₆ alkyl;
Q is >N—R¹³ or 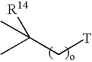
wherein:
o is 0, 1 or 2;
T is —N(R¹⁵)(R¹⁶) or hydroxyl;
R¹³, R¹⁵, and R¹⁶ are independently hydrogen or C₁₋₆ alkyl;
R¹⁴ is hydrogen, aryl or hetaryl;
G is —O—(CH₂)—R¹⁷, 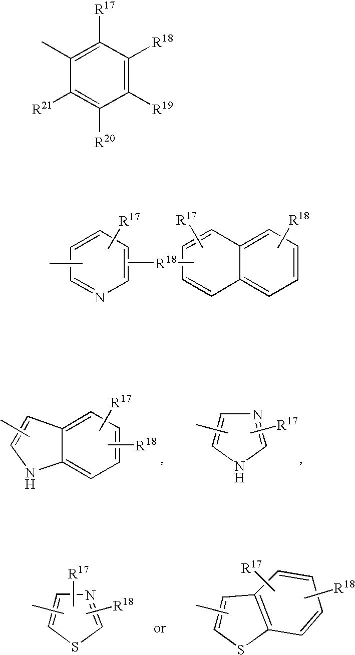
wherein:
R¹⁷, R¹⁸, R¹⁹, R²⁰ and R²¹ independently are hydrogen, halogen, aryl, hetaryl, C₁₋₆-alkyl or C₁₋₆-alkoxy;
K is 0, 1 or 2;
J is —O—(CH₂)ₗ—R²², 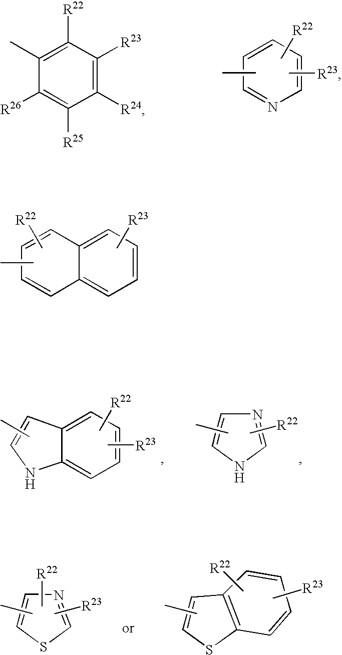
wherein:
R²², R²³, R²⁴, R²⁵ and R²⁶ independently are hydrogen, halogen, aryl, hetaryl, C₁₋₆-alkyl or C₁₋₆-alkoxy;
l is 0, 1 or 2;
a is 0, 1, or 2;
b is 0, 1, or 2;
c is 0, 1, or 2;
d is 0 or 1;
e is 0, 1, 2, or 3;
f is 0 or 1;
R⁵ is hydrogen or C₁₋₆-alkyl optionally substituted with one or more hydroxyl, aryl or hetaryl;
R⁶ and R⁷ are independently hydrogen or C₁₋₆-alkyl, optionally substituted with one or more halogen, amino, hydroxyl, aryl, or hetaryl;
R⁸ is hydrogen or C₁₋₆-alkyl, optionally substituted with one or more halogen, amino, hydroxyl, aryl, or hetaryl;
R⁶ and R⁷ or R⁶ and R⁸ or R⁷ and R⁸ can optionally form —(CH₂)ᵢ—U—(CH₂)ⱼ—, wherein i and j independently are 1, 2 or 3 and U is —O—, —S—, or a valence bond;
M is arylene, hetarylene, —O—, —S— or —CR²⁷═CR²⁸—;
R²⁷ and R²⁸ are independently hydrogen or C₁₋₆-alkyl, optionally substituted with one or more aryl or hetaryl;
or a pharmaceutically acceptable salt thereof.

14. The method of claim 13, wherein the growth hormone secretagogue is represented by Formula V: 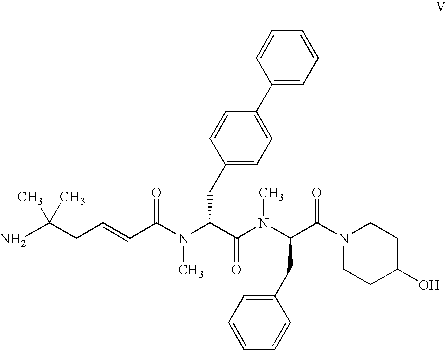
or a pharmaceutically acceptable salt, solvate or hydrate thereof.

15. The method of claim 13, wherein the growth hormone secretagogue is represented by Formula VI: 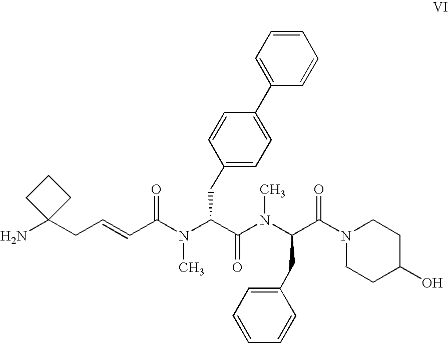
a pharmaceutically acceptable salt, solvate or hydrate thereof.

16. The method of claim 1, wherein the growth hormone secretagogue is administered orally.

17. The method of claim 1, wherein the subject is a human.

18. The method of claim 1, further comprising administering a therapeutically effective amount of a second growth hormone secretagogue, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, bile acid sequestering agent or a combination thereof.

19. The method of claim 18, wherein the growth hormone secretagogue activates the GHS-R1a receptor and the second growth hormone secretagogue activates the GHRH receptor.

20. The method of claim 19, further comprising administering a therapeutically effective amount of a HMG CoA reductase inhibitor.

21. The method of claim 20, wherein the HMG CoA reductase inhibitor is selected from the group consisting of: lovastatin, pravastatin, simvastatin, fluvastatin, atorvastatin, rosuvastatin, cerivastatin or a combination thereof.

22. The method of claim 18, wherein the ACAT inhibitor is selected from the group consisting of: avasimibe, FCE 27677, RP 73163 or a combination thereof.

23. The method of claim 18, wherein the CETP inhibitor is selected from the group consisting of: JTT-705, torcetrapib or a combination thereof.

24. The method of claim 18, wherein the anti-inflammatory agent is selected from the group consisting of: salicylic acid, aspirin, methyl salicylate, diflunisal, salsalate, olsalazine, sulfasalazine, acetaminophen sulindac, etodolac,tolmetin, ketorolac, diclofenac, ibuprofen, naproxen, fenoprofen, ketoprofen, flurbiprofen, oxaprozin, indomethacin, piroxicam, celecoxib, rofecoxib or a combination thereof.

25. The method of claim 18, wherein the ACE inhibitor is selected from the group consisting of: captopril, benazepril, enalapril, fosinopril, lisinopril, quinapril, ramipril, imidapril, perindopril erbumine, trandolapril or a combination thereof.

26. The method of claim 18, wherein the Beta blocker is selected from the group consisting of: sotalol, timolol, esmolol, careolol, carvedilol, nadolol, propanolol, betaxolol, penbutolol, metoprolol, acebutolol, atenolol, labetolol, pindolol or bisoprolol or a combination thereof.

27. The method of claim 18, wherein the cholesterol absorption inhibitor is selected from the group consisting of: ezetimibe, tiqueside, pamaqueside or a combination thereof.

28. The method of claim 18, wherein the nicotonic acid is selected from the group consisting of: niacin, niceritrol or a combination thereof.

29. The method of claim 18, wherein the fibric acid derivative is selected from the group consisting of: clofibrate, gemfibrozil, fenofibrate, ciprofibrate, bezafibrate or a combination thereof.

30. The method of claim 18, wherein the bile acid sequestering agent is selected from the group consisting of: cholestyramine, colestipol or a combination thereof.

31. A method of reducing C-reactive protein in a subject in need thereof suffering from an inflammatory disease or disorder comprising administering a therapeutically effective amount of at least one growth hormone secretagogue compound or a pharmaceutically acceptable salt, hydrate or solvate thereof.

32. The method of claim 31, wherein the inflammatory disease or disorder is selected from the group consisting of: inflammatory conditions of a joint; rheumatoid arthritis; psoriatic arthritis; systemic lupus erythematosus; Sjorgren's syndrome; lung diseases; ARDS; acute pancreatitis; ALS; Alzheimer's disease; cachexia; anorexia; asthma; atherosclerosis; chronic fatigue syndrome; diabetes; insulin diabetes; glomerulonephritis; graft versus host rejection; hemohorragic shock; hyperalgesia; inflammatory bowel disease; multiple sclerosis; myopathies; muscle protein metabolism; osteoporosis; Parkinson's disease; pain; pre-term labor; psoriasis; septic shock; cardiac, allograft; vasculopathy; side effects from radiation therapy; temporal mandibular joint disease; tumor metastasis; or an inflammatory condition resulting from strain, sprain, cartilage damage, trauma such as burn, orthopedic surgery, infection or other disease processes.

33. The method of claim 31, wherein the growth hormone secretagogue compound is represented by the structural Formula I: 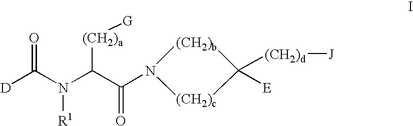
wherein:
R¹ is hydrogen, or C₁₋₆-alkyl optionally substituted with one or more aryl or hetaryl;
a and d are independently 0, 1, 2 or 3;
b and c are independently 0, 1, 2, 3, 4 or 5, provided that b+c is 3, 4 or 5;
D is R²—NH—(CR³R⁴)ₑ—(CH₂)_{f}—M—(CHR⁵)_{g}—(CH₂)ₕ—
wherein:
R², R³, R⁴ and R⁵ are independently hydrogen or C₁₋₆ alkyl optionally substituted with one or more halogen, amino, hydroxyl, aryl or hetaryl; or
R² and R³ or R² and R⁴ or R³ and R⁴ can optionally form —(CH₂)ᵢ—U—(CH₂)ⱼ—, wherein i and j are independently 1 or 2 and U is —O—, —S— or a valence bond;
h and f are independently 0, 1, 2, or 3;
g and e are independently 0 or 1;
M is a valence bond, —CR⁶═CR⁷—, arylene, hetarylene, —O— or —S—;
R⁶ and R⁷ are independently hydrogen, or C₁₋₆-alkyl optionally substituted with one or more aryl or hetaryl;
G is —O—(CH₂)ₖ—R⁸, 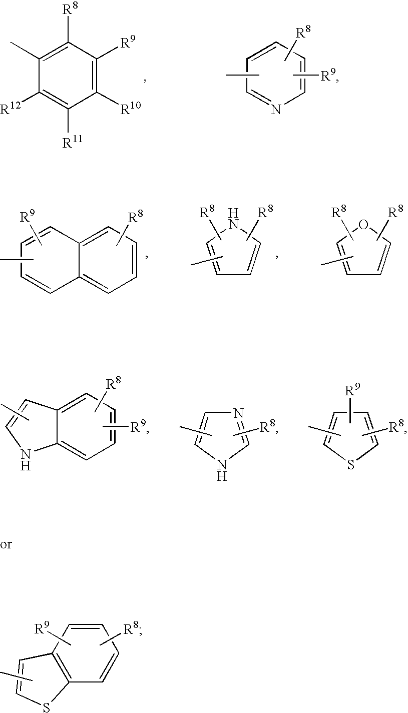
J is —O—(CH₂)ₗ—R¹³, 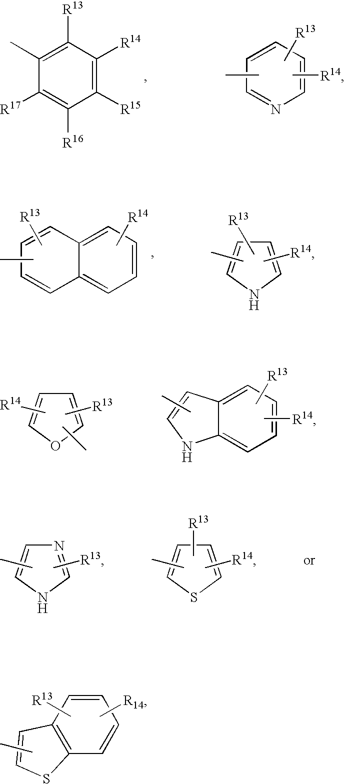
wherein:
R⁸,R⁹,R¹⁰, R¹¹, R¹², R¹³ R¹⁴ R¹⁵ R¹⁶ and R¹⁷ independently are hydrogen, halogen, aryl, hetaryl, C₁₋₆-alkyl or C₁₋₆-alkoxy;
k and 1 are independently 0, 1 or 2;
E is —CONR¹⁸R¹⁹, —COOR¹⁹, —(CH₂)ₘ—NR¹⁸SO₂R²⁰, —(CH₂)ₘ—NR¹⁸—COR²⁰, —(CH₂)ₘ—OR¹⁹, —(CH₂)ₘ—OCOR²⁰, —CH(R¹⁸)R¹⁹, —(CH₂)ₘ—NR¹⁸—CS—NR¹⁹R²¹ or —(CH₂)ₘ—NR¹⁸—CO—NR¹⁹R²¹; or
E is —CONR²²NR²³R²⁴, wherein R²² is hydrogen, C₁₋₆-alkyl optionally substituted with one or more aryl or hetaryl, or aryl or hetaryl optionally substituted with one or more C₁₋₆-alkyl; R²³ is C₁₋₆-alkyl optionally substituted with one or more aryl or hetaryl, or C₁₋₇-acyl; and R²⁴ is hydrogen, C₁₋₆-alkyl optionally substituted with one or more aryl or hetaryl; or aryl or hetaryl optionally substituted with one or more C₁₋₆-alkyl; or
R²² and R²³ together with the nitrogen atoms to which they are attached can form a heterocyclic system optionally substituted with one or more C₁₋₆-alkyl, halogen, amino, hydroxyl, aryl or hetaryl; or
R²² and R²⁴ together with the nitrogen atoms to which they are attached can form a heterocyclic system optionally substituted with one or more C₁₋₆-alkyl, halogen, amino, hydroxyl, aryl or hetaryl; or
R²³ and R²⁴ together with the nitrogen atom to which they are attached can form a heterocyclic system optionally substituted with one or more C₁₋₆-alkyl, halogen, amino, hydroxyl, aryl or hetaryl;
wherein m is 0, 1, 2 or 3,
R¹⁸, R¹⁹ and R²¹ independently are hydrogen or C₁₋₆-alkyl optionally substituted with halogen, —N(R²⁵)R²⁶, wherein R²⁵ and R²⁶ are independently hydrogen or C₁₋₆ alkyl; hydroxyl, C₁₋₆-alkoxy, C₁₋₆-alkoxycarbonyl, C₁₋₆-alkylcarbonyloxy or aryl; or R¹⁹ is 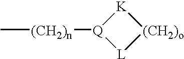
wherein
Q is —CH< or —N<,
K and L are independently —CH₂—, —CO—, —O—, —S—, —NR²⁷— or a valence bond, where R²⁷ is hydrogen or C₁₋₆ alkyl;
n and o are independently 0, 1, 2, 3 or 4;
R²⁰ is C₁₋₆ alkyl, aryl or hetaryl;
or a pharmaceutically acceptable salt thereof;
with the proviso that if M is a valence bond then E is —CONR²²NR²³R²⁴.

34. The method of claim 33, wherein the growth hormone secretaogue is represented by Formula II: 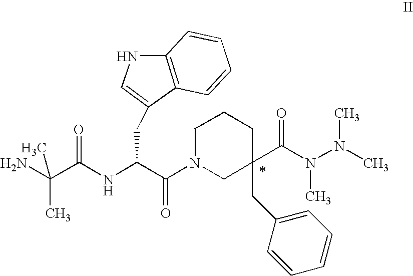
or a pharmaceutically acceptable salt, hydrate or solvate thereof.

35. The method of claim 33, wherein the growth hormone secretagogue is represented by Formula III: 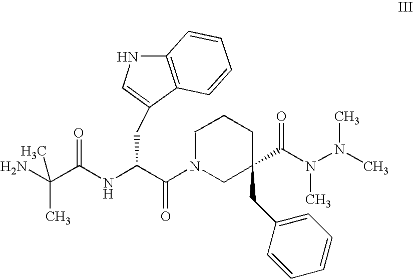
or a pharmacuetically acceptable salt thereof.

36. The method of claim 31, wherein the growth hormone secretagogue is represented by Formula IV: 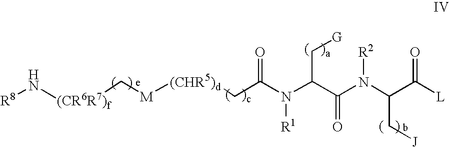
wherein
R¹ is hydrogen or C₁₋₆-alkyl;
R² is hydrogen or C₁₋₆-alkyl;
L is 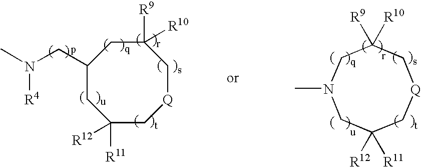
wherein
R⁴ is hydrogen or C₁₋₆ alkyl;
p is 0 or 1;
q, s, t, u are independently 0, 1, 2, 3, or 4;
r is 0 or 1;
the sum q+r+s+t+u is 0, 1, 2, 3, or 4;
R⁹, R¹⁰, R¹¹, and R¹² are independently hydrogen or C₁₋₆ alkyl;
Q is >N—R¹³ or 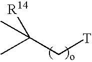
wherein:
o is 0, 1 or 2;
T is —N(R¹⁵)(R¹⁶) or hydroxyl;
R¹³, R¹⁵, and R¹⁶ are independently hydrogen or C₁₋₆ alkyl;
R¹⁴ is hydrogen, aryl or hetaryl;
G is —O—(CH₂)—R¹⁷, 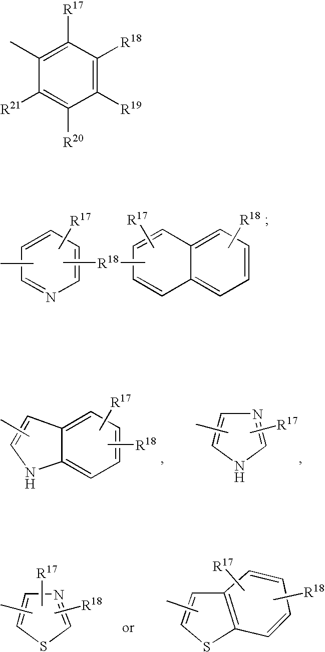
wherein:
R¹⁷, R¹⁸, R¹⁹, R²⁰ and R²¹ independently are hydrogen, halogen, aryl, hetaryl, C₁₋₆-alkyl or C₁₋₆-alkoxy;
K is 0, 1 or 2;
J is —O—(CH₂)ₗ—R²², 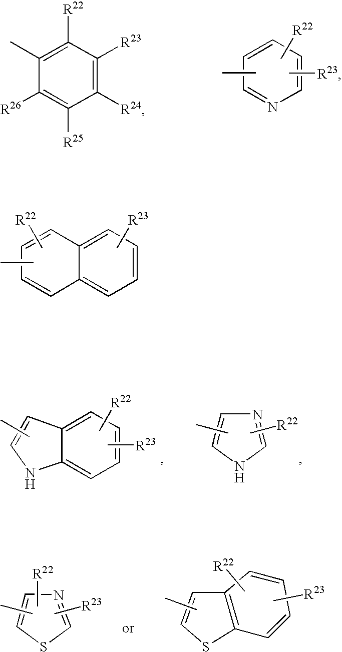
wherein:
R²², R²³, R²⁴, R²⁵ and R²⁶ independently are hydrogen, halogen, aryl, hetaryl, C₁₋₆-alkyl or C₁₋₆-alkoxy;
l is 0, 1 or 2;
a is 0, 1, or 2;
b is 0, 1, or 2;
c is 0, 1, or 2;
d is or 1;
e is 0, 1, 2, or 3;
f is 0 or 1;
R⁵ is hydrogen or C₁₋₆-alkyl optionally substituted with one or more hydroxyl, aryl or hetaryl;
R⁶ and R⁷ are independently hydrogen or C₁₋₆-alkyl, optionally substituted with one or more halogen, amino, hydroxyl, aryl, or hetaryl;
R⁸ is hydrogen or C₁₋₆-alkyl, optionally substituted with one or more halogen, amino, hydroxyl, aryl, or hetaryl;
R⁶ and R⁷ or R⁶ and R⁸ or R⁷ and R⁸ can optionally form —(CH₂)ᵢ—U—(CH₂)ⱼ—, wherein i and j independently are 1, 2 or 3 and U is —O—, —S—, or a valence bond;
M is arylene, hetarylene, —O—, —S— or —CR²⁷═CR²⁸—;
R²⁷ and R²⁸ are independently hydrogen or C₁₋₆-alkyl, optionally substituted with one or more aryl or hetaryl;
or a pharmaceutically acceptable salt thereof.

37. The method of claim 36, wherein the growth hormone secretagogue is represented by Formula V: 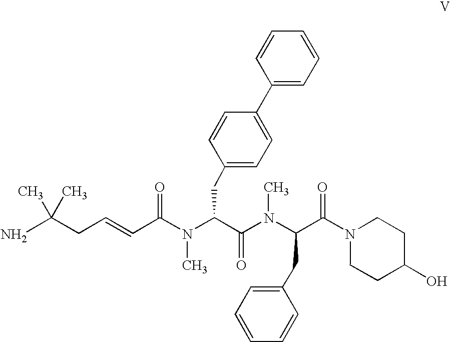
or a pharmaceutically acceptable salt, solvate or hydrate thereof.

38. The method of claim 36, wherein the growth hormone secretagogue is represented by Formula VI: 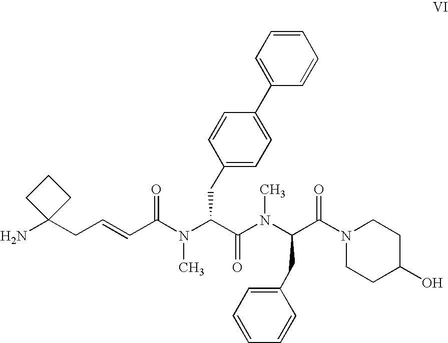
or a pharmaceutically acceptable salt, solvate or hydrate thereof.

39. The method of claim 31, wherein the growth hormone secretagogue is administered orally.

40. The method of claim 31, wherein the subject is a human.

41. The method of claim 31, further comprising administering a therapeutically effective amount of a second growth hormone secretagogue, a HMG CoA reductase inhibitor, an ACAT inhibitor, a CETP inhibitor, an anti-inflammatory agent, an ACE inhibitor, a Beta blocker, a cholesterol absorption inhibitor, a nicotonic acid, a fibric acid derivative, bile acid sequestering agent or a combination thereof.

42. The method of claim 41, wherein the growth hormone secretagogue activates the GHS-R1a receptor and the second growth hormone secretagogue activates the GHRH receptor.

43. The method of claim 42, further comprising administering a therapeutically effective amount of a HMG CoA reductase inhibitor.

44. The method of claim 43, wherein the HMG CoA reductase inhibitor is selected from the group consisting of: lovastatin, pravastatin, simvastatin, fluvastatin, atorvastatin, rosuvastatin, cerivastatin or a combination thereof.

45. The method of claim 41, wherein the ACAT inhibitor is selected from the group consisting of: avasimibe, FCE 27677, RP 73163 or a combination thereof.

46. The method of claim 41, wherein the CETP inhibitor is selected from the group consisting of: JTT-705, torcetrapib or a combination thereof.

47. The method of claim 41, wherein the anti-inflammatory agent is selected from the group consisting of: salicylic acid, aspirin, methyl salicylate, diflunisal, salsalate, olsalazine, sulfasalazine, acetaminophen sulindac, etodolac, tolmetin, ketorolac, diclofenac, ibuprofen, naproxen, fenoprofen, ketoprofen, flurbiprofen, oxaprozin, indomethacin, piroxicam, celecoxib, rofecoxib or a combination thereof.

48. The method of claim 41, wherein the ACE inhibitor is selected from the group consisting of: captopril, benazepril, enalapril, fosinopril, lisinopril, quinapril, ramipril, imidapril, perindopril erbumine, trandolapril or a combination thereof.

49. The method of claim 41, wherein the Beta blocker is selected from the group consisting of: sotalol, timolol, esmolol, careolol, carvedilol, nadolol, propanolol, betaxolol, penbutolol, metoprolol, acebutolol, atenolol, labetolol, pindolol or bisoprolol or a combination thereof.

50. The method of claim 41, wherein the cholesterol absorption inhibitor is selected from the group consisting of: ezetimibe, tiqueside, pamaqueside or a combination thereof.

51. The method of claim 41, wherein the nicotonic acid is selected from the group consisting of: niacin, niceritrol or a combination thereof.

52. The method of claim 41, wherein the fibric acid derivative is selected from the group consisting of: clofibrate, gemfibrozil, fenofibrate, ciprofibrate, bezafibrate or a combination thereof.

53. The method of claim 41, wherein the bile acid sequestering agent is selected from the group consisting of: cholestyramine, colestipol or a combination thereof.
